SEQUENCE LISTING (27 sequences)

SEQ ID NO: 1 - DNA, 3600 bp, hybrid
atggagacagacacactcctgctatgggtactgctgctctgggttccaggttccactggtgacgaagatcaggtagatccgcggttaatcgacggtaagcttagccgagatcaataccatgttttgtttgattcctatagagacaatattgctggaaagtcctttcagaatcggctttgtctgcccatgccgattgacgttgtttacacctgggtgaatggcacagatcttgaactactgaaggaactacagcaggtcagagaacagatggaggaggagcagaaagcaatgagagaaatccttgggaaaaacacaacggaacctactaagaagagtgagaagcagttagagtgtttgctaacacactgcattaaggtgccaatgcttgtcctggacccagccctgccagccaacatcaccctgaaggacctgccatctctttatccttcttttcattctgccagtgacattttcaatgttgcaaaaccaaaaaacccttctaccaatgtctcagttgttgtttttgacagtactaaggatgttgaagatgcccactctggactgcttaaaggaaatagcagacagacagtatggaggggctacttgacaacagataaagaagtccctggattagtgctaatgcaagatttggctttcctgagtggatttccaccaacattcaaggaaacaaatcaactaaaaacaaaattgccagaaaatctttcctctaaagtcaaactgttgcagttgtattcagaggccagtgtagcgcttctaaaactgaataaccccaaggattttcaagaattgaataagcaaactaagaagaacatgaccattgatggaaaagaactgaccataagtcctgcatatttattatgggatctgagcgccatcagccagtctaagcaggatgaagacatctctgccagtcgttttgaagataacgaagaactgaggtactcattgcgatctatcgagaggcatgcaccatgggttcggaatattttcattgtcaccaacgggcagattccatcctggctgaaccttgacaatcctcgagtgacaatagtaacacaccaggatgtttttcgaaatttgagccacttgcctacctttagttcacctgctattgaaagtcacgttcatcgcatcgaagggctgtcccagaagtttatttacctaaatgatgatgtcatgtttgggaaggatgtctggccagatgatttttacagtcactccaaaggccagaaggtttatttgacatggcctgtgccaaactgtgccgagggctgcccaggttcctggattaaggatggctattgtgacaaggcttgtaataattcagcctgcgattgggatggtggggattgctctggaaacagtggagggagtcgctatattgcaggaggtggaggtactgggagtattggagttggacagccctggcagtttggtggaggaataaacagtgtctcttactgtaatcagggatgtgcgaattcctggctcgctgataagttctgtgaccaagcatgcaatgtcttgtcctgtgggtttgatgctggcgactgtgggcaagatcattttcatgaattgtataaagtgatccttctcccaaaccagactcactatattattccaaaaggtgaatgcctgccttatttcagctttgcagaagtagccaaaagaggagttgaaggtgcctatagtgacaatccaataattcgacatgcttctattgccaacaagtggaaaaccatccacctcataatgcacagtggaatgaatgccaccacaatacattttaatctcacgtttcaaaatacaaacgatgaagagttcaaaatgcagataacagtggaggtggacacaagggagggaccaaaactgaattctacggcccagaagggttacgaaaatttagttagtcccataacacttcttccagaggcggaaatcctttttgaggatattcccaaagaaaaacgcttcccgaagtttaagagacatgatgttaactcaacaaggagagcccaggaagaggtgaaaattcccctggtaaatatttcactccttccaaaagacgcccagttgagtctcaataccttggatttgcaactggaacatggagacatcactttgaaaggatacaatttgtccaagtcagccttgctgagatcatttctgatgaactcacagcatgctaaaataaaaaatcaagctataataacagatgaaacaaatgacagtttggtggctccacaggaaaaacaggttcataaaagcatcttgccaaacagcttaggagtgtctgaaagattgcagaggttgacttttcctgcagtgagtgtaaaagtgaatggtcatgaccagggtcagaatccacccctggacttggagaccacagcaagatttagagtggaaactcacacccaaaaaaccataggcggaaatgtgacaaaagaaaagcccccatctctgattgttccactggaaagccagatgacaaaagaaaagaaaatcacagggaaagaaaaagagaacagtagaatggaggaaaatgctgaaaatcacataggcgttactgaagtgttacttggaagaaagctgcagcattacacagatagttacttgggctttttgccatgggagaaaaaaaagtatttcctagatcttctcgacgaagaagagtcattgaagacacaattggcctacttcactgatagcaagaatagagccagatacaagagagatacatttgcagattccctcagatatgtaaataaaattctaaatagcaagtttggattcacatcgcggaaagtccctgctcacatgcctcacatgattgaccggattgttatgcaagaactgcaagatatgttccctgaagaatttgacaagacgtcatttcacaaagtgcgccattctgaggatatgcagtttgccttctcttatttttattatctcatgagtgcagtgcagccactgaatatatctcaagtctttgatgaagttgatacagatcaatctggtgtcttgtctgacagagaaatccgaacactggctaccagaattcacgaactgccgttaagtttgcaggatttgacaggtctggaacacatgctaataaattgctcaaaaatgcttcctgctgatatcacgcagctaaataatattccaccaactcaggaatcctactatgatcccaacctgccaccggtcactaaaagtctagtaacaaactgtaaaccagtaactgacaaaatccacaaagcatataaggacaaaaacaaatataggtttgaaatcatgggagaagaagaaatcgcttttaaaatgattcgtaccaacgtttctcatgtggttggccagttggatgacataagaaaaaaccctaggaagtttgtttgcctgaatgacaacattgaccacaatcataaagatgctcagacagtgaaggctgttctcagggacttctatgaatccatgttccccataccttcccaatttgaactgccaagagagtatcgaaaccgtttccttcatatgcatgagctgcaggaatggagggcttatcgagacaaattgaagtag

SEQ ID NO: 2 - protein, 1199 aa, hybrid
METDTLLLWVLLLWVPGSTGDEDQVDPRLIDGKLSRDQYHVLFDSYRDNIAGKSFQNRLCLPMPIDVVYTWVNGTDLELLKELQQVREQMEEEQKAMREILGKNTTEPTKKSEKQLECLLTHCIKVPMLVLDPALPANITLKDLPSLYPSFHSASDIFNVAKPKNPSTNVSVVVFDSTKDVEDAHSGLLKGNSRQTVWRGYLTTDKEVPGLVLMQDLAFLSGFPPTFKETNQLKTKLPENLSSKVKLLQLYSEASVALLKLNNPKDFQELNKQTKKNMTIDGKELTISPAYLLWDLSAISQSKQDEDISASRFEDNEELRYSLRSIERHAPWVRNIFIVTNGQIPSWLNLDNPRVTIVTHQDVFRNLSHLPTFSSPAIESHVHRIEGLSQKFIYLNDDVMFGKDVWPDDFYSHSKGQKVYLTWPVPNCAEGCPGSWIKDGYCDKACNNSACDWDGGDCSGNSGGSRYIAGGGGTGSIGVGQPWQFGGGINSVSYCNQGCANSWLADKFCDQACNVLSCGFDAGDCGQDHFHELYKVILLPNQTHYIIPKGECLPYFSFAEVAKRGVEGAYSDNPIIRHASIANKWKTIHLIMHSGMNATTIHFNLTFQNTNDEEFKMQITVEVDTREGPKLNSTAQKGYENLVSPITLLPEAEILFEDIPKEKRFPKFKRHDVNSTRRAQEEVKIPLVNISLLPKDAQLSLNTLDLQLEHGDITLKGYNLSKSALLRSFLMNSQHAKIKNQAIITDETNDSLVAPQEKQVHKSILPNSLGVSERLQRLTFPAVSVKVNGHDQGQNPPLDLETTARFRVETHTQKTIGGNVTKEKPPSLIVPLESQMTKEKKITGKEKENSRMEENAENHIGVTEVLLGRKLQHYTDSYLGFLPWEKKKYFLDLLDEEESLKTQLAYFTDSKNRARYKRDTFADSLRYVNKILNSKFGFTSRKVPAHMPHMIDRIVMQELQDMFPEEFDKTSFHKVRHSEDMQFAFSYFYYLMSAVQPLNISQVFDEVDTDQSGVLSDREIRTLATRIHELPLSLQDLTGLEHMLINCSKMLPADITQLNNIPPTQESYYDPNLPPVTKSLVTNCKPVTDKIHKAYKDKNKYRFEIMGEEEIAFKMIRTNVSHVVGQLDDIRKNPRKFVCLNDNIDHNHKDAQTVKAVLRDFYESMFPIPSQFELPREYRNRFLHMHELQEWRAYRDKLK

SEQ ID NO: 3 - DNA, 5597 bp, Homo sapiens
cggagccgagcgggcgtccgtcgccggagctgcaatgagcggcgcccggaggctgtgacctgcgcgcggcggcccgaccggggcccctgaatggcggctcgctgaggcggcggcggcggcggcggctcaggctcctcggggcgtggcgtggcggtgaaggggtgatgctgttcaagctcctgcagagacaaacctatacctgcctgtcccacaggtatgggctctacgtgtgcttcttgggcgtcgttgtcaccatcgtctccgccttccagttcggagaggtggttctggaatggagccgagatcaataccatgttttgtttgattcctatagagacaatattgctggaaagtcctttcagaatcggctttgtctgcccatgccgattgacgttgtttacacctgggtgaatggcacagatcttgaactactgaaggaactacagcaggtcagagaacagatggaggaggagcagaaagcaatgagagaaatccttgggaaaaacacaacggaacctactaagaagagtgagaagcagttagagtgtttgctaacacactgcattaaggtgccaatgcttgtactggacccagccctgccagccaacatcaccctgaaggacgtgccatctctttatccttcttttcattctgccagtgacattttcaatgttgcaaaaccaaaaaacccttctaccaatgtctcagttgttgtttttgacagtactaaggatgttgaagatgcccactctggactgcttaaaggaaatagcagacagacagtatggagggggtacttgacaacagataaagaagtccctggattagtgctaatgcaagatttggctttcctgagtggatttccaccaacattcaaggaaacaaatcaactaaaaacaaaattgccagaaaatctttcctctaaagtcaaactgttgcagttgtattcagaggccagtgtagcgcttctaaaactgaataaccccaaggattttcaagaattgaataagcaaactaagaagaacatgaccattgatggaaaagaactgaccataagtcctgcatatttattatgggatctgagcgccatcagccagtctaagcaggatgaagacatctctgccagtcgttttgaagataacgaagaactgaggtactcattgcgatctatcgagaggcatgcaccatgggttcggaatattttcattgtcaccaacgggcagattccatcctggctgaaccttgacaatcctcgagtgacaatagtaacacaccaggatgtttttcgaaatttgagccacttgcctacctttagttcacctgctattgaaagtcacattcatcgcatcgaagggctgtcccagaagtttatttacctaaatgatgatgtcatgtttgggaaggatgtctggccagatgatttttacagtcactccaaaggccagaaggtttatttgacatggcctgtgccaaactgtgccgagggctgcccaggttcctggattaaggatggctattgtgacaaggcttgtaataattcagcctgcgattgggatggtggggattgctctggaaacagtggagggagtcgctatattgcaggaggtggaggtactgggagtattggagttggacacccctggcagtttggtggaggaataaacagtgtctcttactgtaatcagggatgtgcgaattcctggctcgctgataagttctgtgaccaagcatgcaatgtcttgtcctgtgggtttgatgctggcgactgtgggcaagatcattttcatgaattgtataaagtgatccttctcccaaaccagactcactatattattccaaaaggtgaatgcctgccttatttcagctttgcagaagtagccaaaagaggagttgaaggtgcctatagtgacaatccaataattcgacatgcttctattgccaacaagtggaaaaccatccacctcataatgcacagtggaatgaatgccaccacaatacattttaatctcacgtttcaaaatacaaacgatgaagagttcaaaatgcagataacagtggaggtggacacaagggagggaccaaaactgaattctacggcccagaagggttacgaaaatttagttagtcccataacacttcttccagaggcggaaatcctttttgaggatattcccaaagaaaaacgcttcccgaagtttaagagacatgatgttaactcaacaaggagagcccaggaagaggtgaaaattcccctggtaaatatttcactccttccaaaagacgcccagttgagtctcaataccttggatttgcaactggaacatggagacatcactttgaaaggatacaatttgtccaagtcagccttgctgagatcatttctgatgaactcacagcatgctaaaataaaaaatcaagctataataacagatgaaacaaatgacagtttggtggctccacaggaaaaacaggttcataaaagcatcttgccaaacagcttaggagtgtctgaaagattgcagaggttgacttttcctgcagtgagtgtaaaagtgaatggtcatgaccagggtcagaatccacccctggacttggagaccacagcaagatttagagtggaaactcacacccaaaaaaccataggcggaaatgtgacaaaagaaaagcccccatctctgattgttccactggaaagccagatgacaaaagaaaagaaaatcacagggaaagaaaaagagaacagtagaatggaggaaaatgctgaaaatcacataggcgttactgaagtgttacttggaagaaagctgcagcattacacagatagttacttgggctttttgccatgggagaaaaaaaagtatttccaagatcttctcgacgaagaagagtcattgaagacacaattggcatacttcactgatagcaaaaatactgggaggcaactaaaagatacatttgcagattccctcagatatgtaaataaaattctaaatagcaagtttggattcacatcgcggaaagtccctgctcacatgcctcacatgattgaccggattgttatgcaagaactgcaagatatgttccctgaagaatttgacaagacgtcatttcacaaagtgcgccattctgaggatatgcagtttgccttctcttatttttattatctcatgagtgcagtgcagccactgaatatatctcaagtctttgatgaagttgatacagatcaatctggtgtcttgtctgacagagaaatccgaacactggctaccagaattcacgaactgccgttaagtttgcaggatttgacaggtctggaacacatgctaataaattgctcaaaaatgcttcctgctgatatcacgcagctaaataatattccaccaactcaggaatcctactatgatcccaacctgccaccggtcactaaaagtctagtaacaaactgtaaaccagtaactgacaaaatccacaaagcatataaggacaaaaacaaatataggtttgaaatcatgggagaagaagaaatcgcttttaaaatgattcgtaccaacgtttctcatgtggttggccagttggatgacataagaaaaaaccctaggaagtttgtttgcctgaatgacaacattgaccacaatcataaagatgctcagacagtgaaggctgttctcagggacttctatgaatccatgttccccataccttcccaatttgaactgccaagagagtatcgaaaccgtttccttcatatgcatgagctgcaggaatggagggcttatcgagacaaattgaagttttggacccattgtgtactagcaacattgattatgtttactatattctcattttttgctgagcagttaattgcacttaagcggaagatatttcccagaaggaggatacacaaagaagctagtcccaatcgaatcagagtatagaagatcttcatttgaaaaccatctacctcagcatttactgagcattttaaaactcagcttcacagagatgtctttgtgatgtgatgcttagcagtttggcccgaagaaggaaaatatccagtaccatgctgttttgtggcatgaatatagcccactgactaggaattatttaaccaacccactgaaaacttgtgtgtcgagcagctctgaactgattttacttttaaagaatttgctcatggacctgtcatcctttttataaaaaggctcactgacaagagacagctgttaatttcccacagcaatcattgcagactaactttattaggagaagcctatgccagctgggagtgattgctaagaggctccagtctttgcattccaaagccttttgctaaagttttgcactttttttttttcatttcccatttttaagtagttactaagttaactagttattcttgcttctgagtataacgaattgggatgtctaaacctatttttatagatgttatttaaataatgcagcaatatcacctcttattgacaatacctaaattatgagttttattaatatttaagactgtaaatggtcttaaaccactaactactgaagagctcaatgattgacatctgaaatgctttgtaattattgacttcagcccctaagaatgctatgatttcacgtgcaggtctaatttcaacaggctagagttagtactacttaccagatgtaattatgttttggaaatgtacatattcaaacagaagtgcctcattttagaaatgagtagtgctgatggcactggcacattacagtggtgtcttgtttaatactcattggtatattccagtagctatctctctcagttggtttttgatagaacagaggccagcaaactttctttgtaaaaggctggttagtaaattattgcaggccacctgtgtctttgtcatacattcttcttgctgttgtttagtttgttttttttcaaacaaccctctaaaaatgtaaaaaccatgtttagcttgcagctgtacaaaaactgcccaccagccagatgtgaccctcaggccatcatttgccaatcactgagaattatttttgttgttgttgttgttgttgtttttgagacagagtctctctctgttgcccaggctggagtgcagtggcgcaatctcagctcactgcaacctccgcctcccgggttcaagcagttctgtctcagccttctgagtagctgggactacaggtgcatgccaccacaccctgctaatttttgtatttttagtagagacgggggttccaccatattggtcaggcttatcttgaactcctgacctcaggtgatccacctgcctctgcctcccaaagtgctgagattacaggcataagccagtgcacccagccgagaattagtatttttatgtatggttaaaccttggcgtctagccatattttatgtcataatacaatggatttgtgaagagcagattccatgagtaactctgacaggtattttagatcatgatctcaacaatattcctcccaaatggcatacatcttttgtacaaagaacttgaaatgtaaatactgtgtttgtgctgtaagagttgtgtatttcaaaaactgaaatctcataaaaagttaaattttgaaaa

SEQ ID NO: 4 - protein, 928 aa, Homo sapiens
MLFKLLQRQTYTCLSHRYGLYVCFLGVVVTIVSAFQFGEVVLEWSRDQYHVLFDSYRDNIAGKSFQNRLCLPMPIDVVYTWVNGTDLELLKELQQVREQMEEEQKAMREILGKNTTEPTKKSEKQLECLLTHCIKVPMLVLDPALPANITLKDVPSLYPSFHSASDIFNVAKPKNPSTNVSVVVFDSTKDVEDAHSGLLKGNSRQTVWRGYLTTDKEVPGLVLMQDLAFLSGFPPTFKETNQLKTKLPENLSSKVKLLQLYSEASVALLKLNNPKDFQELNKQTKKNMTIDGKELTISPAYLLWDLSAISQSKQDEDISASRFEDNEELRYSLRSIERHAPWVRNIFIVTNGQIPSWLNLDNPRVTIVTHQDVFRNLSHLPTFSSPAIESHIHRIEGLSQKFIYLNDDVMFGKDVWPDDFYSHSKGQKVYLTWPVPNCAEGCPGSWIKDGYCDKACNNSACDWDGGDCSGNSGGSRYIAGGGGTGSIGVGHPWQFGGGINSVSYCNQGCANSWLADKFCDQACNVLSCGFDAGDCGQDHFHELYKVILLPNQTHYIIPKGECLPYFSFAEVAKRGVEGAYSDNPIIRHASIANKWKTIHLIMHSGMNATTIHFNLTFQNTNDEEFKMQITVEVDTREGPKLNSTAQKGYENLVSPITLLPEAEILFEDIPKEKRFPKFKRHDVNSTRRAQEEVKIPLVNISLLPKDAQLSLNTLDLQLEHGDITLKGYNLSKSALLRSFLMNSQHAKIKNQAIITDETNDSLVAPQEKQVHKSILPNSLGVSERLQRLTFPAVSVKVNGHDQGQNPPLDLETTARFRVETHTQKTIGGNVTKEKPPSLIVPLESQMTKEKKITGKEKENSRMEENAENHIGVTEVLLGRKLQHYTDSYLGFLPWEKKKYFQDLLDEEESLKTQLAYFTDSKNTGRQLK

SEQ ID NO: 5 - protein, 328 aa, Homo sapiens
DTFADSLRYVNKILNSKFGFTSRKVPAHMPHMIDRIVMQELQDMFPEEFDKTSFHKVRHSEDMQFAFSYFYYLMSAVQPLNISQVFDEVDTDQSGVLSDREIRTLATRIHELPLSLQDLTGLEHMLINCSKMLPADITQLNNIPPTQESYYDPNLPPVTKSLVTNCKPVTDKIHKAYKDKNKYRFEIMGEEEIAFKMIRTNVSHVVGQLDDIRKNPRKFVCLNDNIDHNHKDAQTVKAVLRDFYESMFPIPSQFELPREYRNRFLHMHELQEWRAYRDKLKFWTHCVLATLIMFTIFSFFAEQLIALKRKIFPRRRIHKEASPNRIRV

SEQ ID NO: 6 - DNA, 1219 bp, Homo sapiens
gtagagcgcaggtgcgcggctcgatggcggcggggctggcgcggctcctgttgctcctcgggctctcggccggcgggcccgcgccggcaggtgcagcgaagatgaaggtggtggaggagcccaacgcgtttggggtgaacaacccgttcttgcctcaggccagtcgcctccaggccaagagggatccttcacccgtgtctggacccgtgcatctcttccgactctcgggcaagtgcttcagcctggtggagtccacgtacaagtatgagttctgcccgttccacaacgtgacccagcacgagcagaccttccgctggaacgcctacagtgggatcctcggcatctggcacgagtgggagatcgccaacaacaccttcacgggcatgtggatgagggacggtgacgcctgccgttcccggagccggcagagcaaggtggagctggcgtgtggaaaaagcaaccggctggcccatgtgtccgagccgagcacctgcgtctatgcgctgacgttcgagacccccctcgtctgccacccccacgccttgctagtgtacccaaccctgccagaggccctgcagcggcagtgggaccaggtagagcaggacctggccgatgagctgatcaccccccagggccatgagaagttgctgaggacactttttgaggatgctggctacttaaagaccccagaagaaaatgaacccacccagctggagggaggtcctgacagcttggggtttgagaccctggaaaactgcaggaaggctcataaagaactctcaaaggagatcaaaaggctgaaaggtttgctcacccagcacggcatcccctacacgaggcccacagaaacttccaacttggagcacttgggccacgagacgcccagagccaagtctccagagcagctgcggggtgacccaggactgcgtgggagtttgtgaccttgtggtgggagagcagaggtggacgcggccgagagccctacagagaagctggctggtaggacccgcaggaccagctgaccaggcttgtgctcagagaagcagacaaaacaaagattcaaggttttaattaattcccatactgataaaaataactccatgaattctgtaaaccattgcataaatgctatagtgtaaaaaaatttaaacaagtgttaactttaaacagttcgctacaagtaaatgattataaatactaaaaaaaaaaaaaaaaaaaa

SEQ ID NO: 7 - protein, 305 aa, Homo sapiens
MAAGLARLLLLLGLSAGGPAPAGAAKMKVVEEPNAFGVNNPFLPQASRLQAKRDPSPVSGPVHLFRLSGKCFSLVESTYKYEFCPFHNVTQHEQTFRWNAYSGILGIWHEWEIANNTFTGMWMRDGDACRSRSRQSKVELACGKSNRLAHVSEPSTCVYALTFETPLVCHPHALLVYPTLPEALQRQWDQVEQDLADELITPQGHEKLLRTLFEDAGYLKTPEENEPTQLEGGPDSLGFETLENCRKAHKELSKEIKRLKGLLTQHGIPYTRPTETSNLEHLGHETPRAKSPEQLRGDPGLRGSL

SEQ ID NO: 8 - DNA, 5229 bp, Mus musculus
ggcggtgaaggggtgatgctgttcaagctcctgcagagacagacctatacctgcctatcccacaggtatgggctctacgtctgcttcgtgggcgtcgttgtcaccatcgtctcggctttccagttcggagaggtggttctggaatggagccgagatcagtaccatgttttgtttgattcctacagagacaacattgctgggaaatcctttcagaatcggctctgtctgcccatgccaatcgacgtggtttacacctgggtgaatggcactgaccttgaactgctaaaggagctacagcaggtccgagagcacatggaggaagagcagagagccatgcgggaaaccctcgggaagaacacaaccgaaccgacaaagaagagtgagaagcagctggaatgtctgctgacgcactgcattaaggtgcccatgcttgttctggacccggccctgccagccaccatcaccctgaaggatctgccaaccctttacccatctttccacgcgtccagcgacatgttcaatgttgcgaaaccaaaaaatccgtctacaaatgtccccgttgtcgtttttgacactactaaggatgttgaagacgcccatgctggaccgtttaagggaggccagcaaacagatgtttggagagcctacttgacaacagacaaagacgcccctggcttagtgctgatacaaggcttggcgttcctgagtggattcccaccgaccttcaaggagacgagtcaactgaagacaaagctgccaagaaaagctttccctctaaaaataaagctgttgcggctgtactcggaggccagtgtcgctcttctgaaattgaataatcccaagggtttccaagagctgaacaagcagaccaagaagaacatgaccatcgatgggaaggaactgaccatcagccctgcgtatctgctgtgggacctgagtgccatcagccagtccaagcaggatgaggacgcgtctgccagccgctttgaggataatgaagagctgaggtactcgctgcgatctatcgagagacacgcgccatgggtacggaatattttcattgtcaccaacgggcagattccatcctggctgaaccttgacaaccctcgagtgaccatagtgacccaccaggacattttccaaaatctgagccacttgcctactttcagttcccctgctattgaaagtcacattcaccgcatcgaagggctgtcccagaagtttatttatctaaatgacgatgtcatgttcggtaaggacgtctggccggacgatttttacagccactccaaaggtcaaaaggtttatttgacatggcctgtgccaaactgtgcagagggctgcccgggctcctggataaaggacggctattgtgataaggcctgtaatacctcaccctgtgactgggatggcggaaactgctctggtaatactgcagggaaccggtttgttgcaagaggtgggggtaccgggaatattggagctggacagcactggcagtttggtggaggaataaacaccatctcttactgtaaccaaggatgtgcaaactcctggctggctgacaagttctgtgaccaagcctgtaacgtcttatcctgcgggtttgatgctggtgactgtggacaagatcattttcatgaattgtataaagtaacacttctcccaaaccagactcactatgttgtccccaaaggtgaatacctgtcttatttcagctttgcaaacatagccagaaaaagaattgaagggacctacagcgacaaccccatcatccgccacgcgtccattgcaaacaagtggaaaaccctacacctgataatgcccggggggatgaacgccaccacgatctattttaacctcactcttcaaaacgccaacgacgaagagttcaagatccagatagcagtagaggtggacacgagggaggcgcccaaactgaattctacaacccagaaggcctatgaaagtttggttagcccagtgacacctcttcctcaggctgacgtcccttttgaagatgtccccaaagagaaacgcttccccaagatcaggagacatgatgtaaatgcaacagggagattccaagaggaggtgaaaatcccccgggtaaatatttcactccttcccaaagaggcccaggtgaggctgagcaacttggatttgcaactagaacgtggagacatcactctgaaaggatataacttgtccaagtcagccctgctaaggtctttcctggggaattcactagatactaaaataaaacctcaagctaggaccgatgaaacaaaaggcaacctggaggtcccacaggaaaacccttctcacagacgtccacatggctttgctggtgaacacagatcagagagatggactgccccagcagagacagtgaccgtgaaaggccgtgaccacgctttgaatccacccccggtgttggagaccaatgcaagattggcccagcctacactaggcgtgactgtgtccaaagagaacctttcaccgctgatcgttcccccagaaagccacttgccaaaagaagaggagagtgacagggcagaaggcaatgctgtacctgtaaaggagttagtgcctggcagacggttgcagcagaattatccaggctttttgccctgggagaaaaaaaagtatttccaagaccttcttgatgaggaagagtcattgaagacccagttggcgtactttacagaccgcaaacataccgggaggcaactaaaagatacatttgcagactccctccgatacgtcaataaaattctcaacagcaagtttggattcacatccaggaaagtccctgcacacatgccgcacatgattgacaggatcgttatgcaagaactccaagatatgttccctgaagaatttgacaagacttcatttcacaaggtgcgtcactctgaggacatgcagtttgccttctcctacttttattacctcatgagtgcagttcagcccctcaatatttcccaagtctttcatgaagtagacacagaccaatctggtgtcttgtctgatagggaaatccgaacwctggccacgagaattcacgacctacctttaagcttgcaggatttgacaggtttggaacacatgttaataaattgctcaaaaatgctccccgctaatatcactcaactcaacaacatcccaccgactcaggaagcatactacgaccccaacctgcctccggtcactaagagtcttgtcaccaactgtaagccagtaactgacaagatccacaaagcctataaagacaagaacaaatacaggtttgaaatcatgggagaggaagaaatcgctttcaagatgatacgaaccaatgtttctcatgtggttggtcagttggatgacatcagaaaaaaccccaggaagttcgtttgtctgaatgacaacattgaccacaaccataaagatgcccggacagtgaaggctgtcctcagggacttctatgagtccatgtttcccataccttcccagtttgagctgccaagagagtatcggaaccgctttctgcacatgcatgagctccaagaatggcgggcatatcgagacaagctgaagttttggacccactgcgtactagcaacgttgattatatttactatattctcattttttgctgaacagataattgctctgaagcgaaagatatttcccaggaggaggatacacaaagaagctagtccagaccgaatcagggtgtagaagatcttcatttgaaagtcacctaccttagcatctgtgaacatctccctcctcgacaccacagcggagtccctgtgatgtggcacagaggcagcctcgtggggagaagggacatcgtgcagaccgggttcttctgcaatgggaagagagcccactgacctggaattattcagcacactaagaacctgtgtcaatagcttgtacagcttgtacttttaaaggatttgccgaaggacctgtcggcttgttgacaaaccctccctgacaagctgctggtttcttcccccagttactgcagactgagaaaccagtccatcttgaaagcaagtgcggaggggccccagtctttgcattccaaagctttccagcataatttctggcttgtctcctcctttgatccatttcccatttttttttaaaaaacaataagtggctactaagttagtcattctcacttctcaaaataacaaatcaggatgtcaaaacatttgtatagatcttatttaaataatatagaacgattacttctttagcctatctaaattattgatttttattaacagtcaagtggtcttgaaccgctaacaactactgaagagctcgagattgacgttgaaagtgctttgagcttgtttaactcattccccaagaatactgtgacctcgtgtgcgggcctgattgcgaagggctagtgtcacgtagcagtgctgctcaccggatgtaattatgtcgtggaaatgtacatacagacaaaagtgcctcacttcagaaatgagtagtgctgatggcaccagcgagtgatggtgtccatttggaaacccatgataccttccaatgcccaccctgcttactttatacagagcaggggttaaccaacttctgtcaaagaacagtaaagaacttgagatacatccatctttgtcaaatagttttccttgctaacatttattattgttggtgttttgggaggtttattttattttattgctttgttatttttcaagacggggattctctgtgtagctctggctgtttggtaattcactctaaagaccaggctggccttgaacttagagattcacctgcttctgcttcctgaatggtaggacatgtgcccacattgcctacccaccccccttttggggggggtgagcaactcaataaaaagatgaaaacctgctttagtttgcagctatacaaaagcagcaggcctcagccagacttgacccccggggccattgttggcccacgggagaatcatttttgacgtgggtaagcaaaccctgatattggtcatgctgtgttatgtcattatgtggtggttttgaattttggaagatattttcagtcatgatttcagtagtattcctccaaaatggcacacatttttgtaataagaacttgaaatgtaaatattgtgtttgtgctgtaaattttgtgtatttcaaaaactgaagtttcataaaaaaacacacttattggaaaaaaaaaaaaaaaaaaaaa

SEQ ID NO: 9 - protein, 908 aa, Mus musculus
MLFKLLQRQTYTCLSHRYGLYVCFVGVVVTIVSAFQFGEVVLEWSRDQYHVLFDSYRDNIAGKSFQNRLCLPMPIDVVYTWVNGTDLELLKELQQVREHMEEEQRAMRETLGKNTTEPTKKSEKQLECLLTHCIKVPMLVLDPALPATITLKDLPTLYPSFHASSDMFNVAKPKNPSTNVPVVVFDTTKDVEDAHAGPFKGGQQTDVWRAYLTTDKDAPGLVLIQGLAFLSGFPPTFKETSQLKTKLPRKAFPLKIKLLRLYSEASVALLKLNNPKGFQELNKQTKKNMTIDGKELTISPAYLLWDLSAISQSKQDEDASASRFEDNEELRYSLRSIERHAPWVRNIFIVTNGQIPSWLNLDNPRVTIVTHQDIFQNLSHLPTFSSPAIESHIHRIEGLSQKFIYLNDDVMFGKDVWPDDFYSHSKGQKVYLTWPVPNCAEGCPGSWIKDGYCDKACNTSPCDWDGGNCSGNTAGNRFVARGGGTGNIGAGQHWQFGGGINTISYCNQGCANSWLADKFCDQACNVLSCGFDAGDCGQDHFHELYKVTLLPNQTHYVVPKGEYLSYFSFANIARKRIEGTYSDNPIIRHASIANKWKTLHLIMPGGMNATTIYFNLTLQNANDEEFKIQIAVEVDTREAPKLNSTTQKAYESLVSPVTPLPQADVPFEDVPKEKRFPKIRRHDVNATGRFQEEVKIPRVNISLLPKEAQVRLSNLDLQLERGDITLKGYNLSKSALLRSFLGNSLDTKIKPQARTDETKGNLEVPQENPSHRRPHGFAGEHRSERWTAPAETVTVKGRDHALNPPPVLETNARLAQPTLGVTVSKENLSPLIVPPESHLPKEEESDRAEGNAVPVKELVPGRRLQQNYPGFLPWEKKKYFQDLLDEEESLKTQLAYFTDRKHTGRQLK

SEQ ID NO: 10 - protein, 328 aa, Mus musculus
DTFADSLRYVNKILNSKFGFTSRKVPAHMPHMIDRIVMQELQDMFPEEFDKTSFHKVRHSEDMQFAFSYFYYLMSAVQPLNISQVFHEVDTDQSGVLSDREIRTLATRIHDLPLSLQDLTGLEHMLINCSKMLPANITQLNNIPPTQEAYYDPNLPPVTKSLVTNCKPVTDKIHKAYKDKNKYRFEIMGEEEIAFKMIRTNVSHVVGQLDDIRKNPRKFVCLNDNIDHNHKDARTVKAVLRDFYESMFPIPSQFELPREYRNRFLHMHELQEWRAYRDKLKFWTHCVLATLIIFTIFSFFAEQIIALKRKIFPRRRIHKEASPDRIRV

SEQ ID NO: 11 - DNA, 2070 bp, Mus musculus
  misc_feature (186)..(186): n is a, t, c, or g
gtgagaccctaggagcaatggccgggcggctggctggcttcctgatgttgctggggctcgcgtcgcaggggcccgcgccggcatgtgccgggaagatgaaggtggtggaggagcctaacacattcgggtgagcggatcacggtcctgcggcttggggaccgagcctggctggttcttctgaccttntcaattccataggctgaataacccgttcttgccccaggcaagccgccttcagcccaagagagagccttcagctgtatcccgcaaattaagagaaattaatttcaaacgatttagaaagtattctagccaggcgatgatggcgcacgcctttaatcccagcacttgggaggcagaggcaggcagatttccgagttcaaggccatcagaactgactgtacatcttagtacagtttagcatgtgatcagagatctgaatcacaaagctgggcctgcgtggtaaagcaggtcctttctaataaggttgcagtttagattttctttcttaactcttttattctttgagacagggtttctcaacagtgggtgtcctggaactcacttttgtaaaccaggctgcccttaaactcacaaagctctgtcagcctctgcctcctgagtgctgggattaaaggtccacaccctgttcattcatttttaatttttgagactgggtctcattatgtggccctagacagatactgagagcctcctccacaggaacaagcatgggaatcctgccacagacaaccagttctgtggtctggagatgagtttgtcagtccctaggagttaggtcagcctgcctctgcattcccaataatttaggaaaggagcttggggcgttctggccttgatggttagtgccctcctgccaaccttagcttccagctttaggggtagcagagtttataccgatgctaaactgctgttgtgttcttccccagggcccctgcatctcttcagacttgctggcaagtgctttagcctagtggagtccacgtgagtgccaggctggtgggtggagtgggcggagtctgcagagctcctgatgtgcctgtgtttcccaggtacaagtatgaattctgccctttccacaacgtcacccagcacgagcagaccttccgctggaatgcctacagcgggatccttggcatctggcatgagtgggaaatcatcaacaataccttcaagggcatgtggatgactgatggggactcctgccactcccggagccggcagagcaaggtggagctcacctgtggaaagatcaaccgactggcccacgtgtctgagccaagcacctgtgtctatgcattgacattcgagacccctcttgtttgccatccccactctttgttagtgtatccaactctgtcagaagccctgcagcagcccttggaccaggtggaacaggacctggcagatgaactgatcacaccacagggctatgagaagttgctaagggtactttttgaggatgctggctacttaaaggtcccaggagaaacccatcccacccagctggcaggaggttccaagggcctggggcttgagactctggacaactgtagaaaggcacatgcagagctgtcacaggaggtacaaagactgacgagtctgctgcaacagcatggaatcccccacactcagcccacaggtcagtctgcctgccctggtcagctgccagccactccggggcctgcagcactggggcagatctttattgctacccattctggcagaaaccactcactctcagcacctgggtcagcagctccccataggtgcaatcgcagcagagcatctgcggagtgacccaggactacgtgggaacatcctgtgagcaaggtggccacgaagaatagaaatatcctgagctttgagtgtcctttcacagagtgaacaaaactggtgtggtgtagacacggcttcttttggcatattctagatcagacagtgtcactgacaaacaagagggacctgctggccagcctttgttgtgcccaaagatccagacaaaataaagattcaaagttttaattaaaaaaaaaaaaaaaggaattc

SEQ ID NO: 12 - protein, 307 aa, Mus musculus
MAGRLAGFLMLLGLASQGPAPACAGKMKVVEEPNTFGLNNPFLPQASRLQPKREPSAVSGPLHLFRLAGKCFSLVESTYKYEFCPFHNVTQHEQTFRWNAYSGILGIWHEWEIINNTFKGMWMTDGDSCHSRSRQSKVELTCGKINRLAHVSEPSTCVYALTFETPLVCHPHSLLVYPTLSEALQQRLDQVEQDLADELITPQGYEKLLRVLFEDAGYLKVPGETHPTQLAGGSKGLGLETLDNCRKAHAELSQEVQRLTSLLQQHGIPHTQPTETTHSQHLGQQLPIGAIAAEHLRSDPGLRGNIL

SEQ ID NO: 13 - DNA, 460 bp, Rattus rattus
attcccaccaacattcaaggagacgagtcagctgaagacaaaactgccagaaaatctttcttctaaaataaaactgttgcagctgtactcggaggccagcgtcgctcttctgaaattgaataaccccaaaggtttccccgagctgaacaagcagaccaagaagaacatgagcatcagtgggaaggaactggccatcagccctgcctatctgctgtgggacctgagcgccatcagccagtccaagcaggatgaagatgtgtctgccagccgcttcgaggataacgaagagctgaggtactcactgagatctatcgagagacatgattccatgagtcctttatgaattctggccatatcttcaatcatgatctcagtagtattcctctgaaatggcacacatttttctaatgagaacttgaaatgtaaatattgtgtttgtgctgtaaattttgtgtatttc

SEQ ID NO: 14 - protein, 113 aa, Rattus rattus
FPPTFKETSQLKTKLPENLSSKIKLLQLYSEASVALLKLNNPKGFPELNKQTKKNMSISGKELAISPAYLLWDLSAISQSKQDEDVSASRFEDNEELRYSLRSIERHDSMSPL

SEQ ID NO: 15 - DNA, 1105 bp, Drosophila melanogaster
  misc_feature (903)..(903): n is a, g, t, or c
ctgcaggaattcggcacgaggcggttcgatgacaagaatgagctgcggtactctctgaggtccctggaaaaacacgccgcatggatcaggcatgtgtacatagtaaccaatggccagattccaagttggctggatctcagctacgaaagggtcacggtggtgccccacgaagtcctggctcccgatcccgaccagctgcccaccttctccagctcggccatcgagacatttctgcaccgcataccaaagctgtccaagaggttcctctacctcaacgacgacatattcctgggagctccgctgtatccggaggacttgtacactgaagcggagggagttcgcgtgtaccaggcatggatggtgcccggctgcgccttggattgcccctggacgtacataggtgatggagcttgcgatcggcactgcaacattgatgcgtgccaatttgatggaggcgactgcagtgaaactgggccagcgagcgatgcccacgtcattccaccaagcaaagaagtgctcgaggtgcagcctgccgctgttccacaatcaagagtccaccgatttcctcagatgggtctccaaaagctgttcaggcgcagctctgccaattttaaggatgttatgcggcaccgcaatgtgtccacactcaaggaactacgtcgcattgtggagcgttttaacaaggccaaactcatgtcgctgaaccccgaactggagacctccagctccgagccacagacaactcagcgccacgggctgcgcaaggaggattttaagtcttccaccgatatttactctcactcgctgattgccaccaatatgttgctgaatagagcctatggctttaaggcacgccatgtcctggcgcacgtgggcttcctaattgacaaggatattgtggangccatgcaacgacgttttaccagcgaattctngacactggccattaacgctttccgagccccaacagatttgcagtacgcattcgcttactacttctttctaatgagcgaaatccaagtnatgagtgtagangaaatcttcgatgaagtcgacaccggacggtttggncacctggtcggatccagaagtgcgaaccntttta

SEQ ID NO: 16 - protein, 502 aa, Drosophila melanogaster
GTRRFDDKNELRYSLRSLEKHAAWIRHVYIVTNGQIPSWLDLSYERVTVVPHEVLAPDPDQLPTFSSSAIETFLHRIPKLSKRFLYLNDDIFLGAPLYPEDLYTEAEGVRVYQAWMVPGCALDCPWTYIGDGACDRHCNIDACQFDGGDCSETGPASDAHVIPPSKEVLEVQPAAVPQSRVHRFPQMGLQKLFRRSSANFKDVMRHRNVSTLKELRRIVERFNKAKLMSLNPELETSSSEPQTTQRHGLRKEDFKSSTDIYSHSLIATNMLLNRAYGFKARHVLAHVGFLIDKDIVEAMQRRFHQQILDTAHQRFRAPTDLQYAFAYYSFLMSETKVMSVEEIFDEFDTDGSATWSDREVRTFLTRIYQPPLDWSAMRYFEEVVQNCTRNLGMHLKVDTVEHSTLVYERYEDSNLPTITRDLVVRCPLLAEALAANFAVRPKYNFHVSPKRTSHSNFMMLTSNLTEVVESLDRLRRNPRKFNCINDNLDANRGEDNEDGAPS

SEQ ID NO: 17 - DNA, 2183 bp, Homo sapiens
atggcgacctccacgggtcgctggcttctcctccggcttgcactattcggcttcctctgggaagcgtccggcggcctcgactcgggggcctcccgcgacgacgacttgctactgccctatccacgcgcgcgcgcgcgcctcccccgggactgcacacgggtgcgcgccggcaaccgcgagcacgagagttggcctccgcctcccgcgactcccggcgccggcggtctggccgtgcgcaccttcgtgtcgcacttcagggaccgcgcggtggccggccacctgacgcgggccgttgagcccctgcgcaccttctcggtgctggagcccggtggacccggcggctgcgcggcgagacgacgcgccaccgtggaggagacggcgcgggcggccgactgccgtgtcgcccagaacggcggcttcttccgcatgaactcgggcgagtgcctggggaacgtggtgagcgacgagcggcgggtgagcagctccggggggctgcagaacgcgcagttcgggatccgccgcgacgggaccctggtcaccgggtacctgtctgaggaggaggtgctggacactgagaacccatttgtgcagctgctgagtggggtcgtgtggctgattcgtaatggaagcatctacatcaacgagagccaagccacagagtgtgacgagacacaggagacaggttcctttagcaaatttgtgaatgtgatatcagccaggacggccattggccacgaccggaaagggcagctggtgctctttcatgcagacggccatacggagcagcgtggcatcaacctgtgggaaatggcggagttcctgctgaaacaggacgtggtcaacgccatcaacctggatgggggtggctctgccacctttgtgctcaacgggaccttggccagttacccgtcagatcactgccaggacaacatgtggcgctgtccccgccaagtgtccaccgtggtgtgtgtgcacgaaccccgctgccagccgcctgactgccacggccacgggacctgcgtggacgggcactgccaatgcaccgggcacttctggcggggtcccggctgtgatgagctggactgtggcccctctaactgcagccagcacggactgtgcacggagaccggctgccgctgtgatgccggatggaccgggtccaactgcagtgaagagtgtccccttggctggcatgggccgggctgccagaggcgttgtaagtgtgagcaccattgtccctgtgaccccaagactggcaactgcagcgtctccagagtaaagcagtgtctccagccacctgaagccaccctgagggcgggagaactctcctttttcaccaggaccgcctggctagccctcaccctggcgctggccttcctcctgctgatcagcattgcagcaaacctgtccttgctcctgtccagagcagagaggaaccggcgcctgcatggggactatgcataccacccgctgcaggagatgaacggggagcctctggccgcagagaaggagcagccagggggcgcccacaaccccttcaaggactgaagcctcaagctgcccggggtggcacgtcgcgaaagcttgtttccccacggtctggcttctgcaggggaaatttcaaggccactggcgtggaccatctgggtgtcctcaatggcccctgtggggcagccaagttcctgatagcacttgtgcctcagcccctcacctggccacctgccagggcacctgcaaccctagcaataccatgctcgctggagaggctcagctgcctgcttctcgcctgcctgtgtctgctgccgagaagcccgtgcccccgggagggctgccgcactgccaaagagtctccctcctcctggggaaggggctgccaacgaaccagactcagtgaccacgtcatgacagaacagcacatcctggccagcacccctggctggagtgggttaaagggacgagtctgccttcctggctgtgacacgggaccccttttctacagacctcatcactggatttgccaactagaattcgatttcctgtcataggaagctccttggaagaagggatggggggatgaaatcatgtttacagacctgttttgtcatcctgctgccaagaagttttttaatcacttgaataaattgatataataaaaggagccaccaggtggtgtgtggattctg

SEQ ID NO: 18 - protein, 515 aa, Homo sapiens
MATSTGRWLLLRLALFGFLWEASGGLDSGASRDDDLLLPYPRARARLPRDCTRVRAGNREHESWPPPPATPGAGGLAVRTFVSHFRDRAVAGHLTRAVEPLRTFSVLEPGGPGGCAARRRATVEETARAADCRVAQNGGFFRMNSGECLGNVVSDERRVSSSGGLQNAQFGIRRDGTLVTGYLSEEEVLDTENPFVQLLSGVVWLIRNGSIYINESQATECDETQETGSFSKFVNVISARTAIGHDRKGQLVLFHADGHTEQRGINLWEMAEFLLKQDVVNAINLDGGGSATFVLNGTLASYPSDHCQDNMWRCPRQVSTVVCVHEPRCQPPDCHGHGTCVDGHCQCTGHFWRGPGCDELDCGPSNCSQHGLCTETGCRCDAGWTGSNCSEECPLGWHGPGCQRRCKCEHHCPCDPKTGNCSVSRVKQCLQPPEATLRAGELSFFTRTAWLALTLALAFLLLISIAANLSLLLSRAERNRRLHGDYAYHPLQEMNGEPLAAEKEQPGGAHNPFKD

SEQ ID NO: 19 - DNA, 2005 bp, Mus musculus
gtttcccgcgacgatgacctgctgctgccttacccactagcgcgcagacgtccctcgcgagactgcgcccgggtgcgctcaggtagcccagagcaggagagctggcctccgccacctctggccacccacgaaccccgggcgccaagccaccacgcggccgtgcgcaccttcgtgtcgcacttcgaggggcgcgcggtggccggccacctgacgcgggtcgccgatcccctacgcactttctcggtgctggagcccggaggagccgggggctgcggcggcagaagcgccgcggctactgtggaggacacagccgtccgggccggttgccgcatcgctcagaacggtggcttcttccgcatgagcactggcgagtgcttggggaacgtggtgagcgacgggcggctggtgagcagctcagggggactgcagaacgcgcagttcggtatccgacgcgatggaaccatagtcaccgggtcctgtcttgaagaagaggttctggatcccgtgaatccgttcgtgcagctgctgagcggagtcgtgtggctcatccgcaatggaaacatctacatcaacgagagccaagccatcgagtgtgacgagacacaggagacaggttcttttagcaaatttgtgaatgtgatgtcagccaggacagccgtgggtcatgaccgtgaggggcagcttatcctcttccatgctgatggacagacggaacagcgtggccttaacctatgggagatggcagagttcctgcgtcaacaagatgtcgtcaatgccatcaacctggatggaggcggttctgctacttttgtgctcaatgggaccctggccagttacccttcagatcactgccaggacaacatgtggcgctgtccccgccaagtgtccactgtggtgtgtgtgcatgaaccgcgctgccagccacccgactgcagtggccatgggacctgtgtggatggccactgtgaatgcaccagccacttctggcggggcgaggcctgcagcgagctggactgtggcccctccaactgcagccagcatgggctgtgcacagctggctgccactgtgatgctgggtggacaggatccaactgcagtgaagagtgtcctctgggctggtatgggccaggttgccagaggccctgccagtgtgagcaccagtgtttctgtgacccgcagactggcaactgcagcatctcccaagtgaggcagtgtctccagccaactgaggctacgccgagggcaggagagctggcctctttcaccaggaccacctggctagccctcaccctgacactaattttcctgctgctgatcagcactggggtcaacgtgtccttgttcctgggctccagggccgagaggaaccggcacctcgacggggactatgtgtatcacccactgcaggaggtgaacggggaagcgctgactgcagagaaggagcacatggaggaaactagcaaccccttcaaggactgaagagctgccccaacggcatgctccagataatcttgtccctgctcctcacttccacaggggacattgtgaggccactggcatggatgctatgcaccccaccctttgctggccatattcctcctgtccccatgctgtggctcatgccaacctagcaataaggagctctggagagcctgcacctgcctcccgctcgcctatatctgctgcccagaggcctgtctcgcacaggggtctcgccactgccaaagactcccaggaagtcaaagactcccagtaatccactagcaaatggaactctgtaacgccatcataacaagagtggccactctccgcgtgcacaggtatgaaatataaatccttacacacacacacacacacaccctcggctcagccacggcactcgccttttatacagcgtcatcgctggacagccaactagaactctgcatcctgtcacaggaagcacctcataagaaggaatggggagggaaggcagtcgccttgttttcagaccttagccgaattc

SEQ ID NO: 20 - protein, 492 aa, Mus musculus
VSRDDDLLLPYPLARRRPSRDCARVRSGSPEQESWPPPPLATHEPRAPSHHAAVRTFVSHFEGRAVAGHLTRVADPLRTFSVLEPGGAGGCGGRSAAATVEDTAVRAGCRIAQNGGFFRMSTGECLGNVVSDGRLVSSSGGLQNAQFGIRRDGTIVTGSCLEEEVLDPVNPFVQLLSGVVWLIRNGNIYINESQAIECDETQETGSFSKFVNVMSARTAVGHDREGQLILFHADGQTEQRGLNLWEMAEFLRQQDVVNAINLDGGGSATFVLNGTLASYPSDHCQDNMWRCPRQVSTVVCVHEPRCQPPDCSGHGTCVDGHCECTSHFWRGEACSELDCGPSNCSQHGLCTAGCHCDAGWTGSNCSEECPLGWYGPGCQRPCQCEHQCFCDPQTGNCSISQVRQCLQPTEATPRAGELASFTRTTWLALTLTLIFLLLISTGVNVSLFLGSRAERNRHLDGDYVYHPLQEVNGEALTAEKEHMEETSNPFKD

SEQ ID NO: 21 - DNA, 9792 bp, Mus musculus
caggctcgggacttactataacacaggacacttgtcacctgaaagcttgagtcagtcagttattatggtctgtgtgtgagatacaagtgggtgcataggcagtggtgcacacatgtagatcagactttctacagccaattctcttcttcctcctctccatgggttcagggtcttcatctcaggttgcacagcgagttcatttatgtgctgtgccatctcgccagtcgttcctatatcctagaggaaaactagtttcttctggtcaagaggaggaaagagtggagacctgtcattctaagatacccaaaacagggccaggttggggacctgtgcctttaatcccatcacttggggattaggtagaagcaagaggctctagaccagtctacacactgaatttcaagccagcctacctataaatcagagaccctgcttcaaaaataaaattaaacaaaaacgaagataaaccaagctacccaaaacacaagagttaatccagtcagacaggtctagcaaatgctaggatgaaaggtgtgcaccaccacgagtgggctgcaagcctctctctctctctctctctctctctctctctcgtttgttttgtttttcgagacaaggtttctctgtgtagccctggctgtcctggaactcactctgtagaccaggctggcctcgagcttcactcttaaaagttcctcttcctcctcctccatcttttcctcctcttaccccctaggctccttttcctcttcttgtctttcagataaagtctcaagtagtccagactggtctcaaactaactaactagccaagaatagccaacctcttaacttccgattctcctgcctctgctgaatgctggggttgtggcgtgggccaccacttctggtttgtgcaacacagaaggaactagggctttaagcacgagaagcaagttctgtacagacttacacaggcccagcatctgttcttgcaattttctgtaagtttgacataatatgagaataaaaagctatctatctcccttccagccttaccctctctgatggaattcgaatgcgtaatcaaagcacccaacagcctggcctgaaatcacgtggggcaagcccacgtgaccggagcaccaatccaatatggcggcgcccagggggcccgggctgttcctcatacccgcgctgctcggcttactcggggtggcgtggtgcagcttaagcttcgggtgagtgcaagccgccggggccagcctggctggggtccacctttcctgagcgctctcaggcacagccctccgacctcacgatcgccccgtccctgcagggtttcccgcgacgatgacctgctgctgccttacccactagcgcgcagacgtccctcgcgagactgcgcccgggtgcgctcaggtagcccagagcaggagagctggcctccgccacctctggccacccacgaaccccgggcgccaagccaccacgcggccgtgcgcaccttcgtgtcgcacttcgaggggcgcgcggtggccggccacctgacgcgggtcgccgatcccctacgcactttctcggtgctggagcccggaggagccgggggctgcggcggcagaagcgccgcggctactgtggaggacacagccgtccgggccggttgccgcatcgctcagaacggtggcttcttccgcatgagcactggcgagtgcttggggaacgtggtgagcgacgggcggctggtgagcagctcagggggactgcagaacgcgcagttcggtatccgacgcgatggaaccatagtcaccgggtgaggaggcagggagccccggggctgtagagggcaaagggtctctgatgttctttcagagccatgcctccgagtccaggtccctaaccaaacttcctgtctttcttcttccgagtaatgacgctgacaccttccttcctttaagtttattcatgtgccactgaataatctgtgatcaggccgtgtgtggggacttggggaggcgaccgtgagcctgaacacagtttgtgccctagtgaactttgtgtagtattagagaaacatttcgtgttcaacgaagccatggaaccaattggaaatagtgtagagtttatggagcagtcccagacagctagctggaggccttttgctgtcctgataaaaatccaggttagacaaggagcttgttgagggcagcctttggaagtttctgtgtttcttgaaatttgacagcagccagagttgacagcaggcaggcaggagtagaaggtagcgccatctggtgttccagttctcttccaaggttccgttttttgccaaggctgggaagtgggctttccccaactcttctcagcccttggttgcaatttctgggcctgcccatgtatctggttcttcatccttcaacatcagccagtgtcaccactgttgatcttaggttttcacagatcctaaaacttctgccagtgaccagcgcctgcagtttctcttccctggctctgtccttcaacctctctacattccagccatctccctagctcctctcttggactccctttcagacttgttgtcatgatcactgtctcagaacccctattgctcctttacaatggtccactgacctgctcacctcctactttttttttttaaatgtgtgtgcatctgtgtgtgcctgaggggagaccagagtttgatttcaaatgtcttctattctcttttcctccatcttattttctaacacaaaatctgaatctagagatcactggttcagttaacctggctggccggtaaaccccagggccctcctgcttccctctgtccaccccaccccagcactaaggctacagtgtgtgctgttccagccagctttctcatgggtgctgaggatctgaacgcaggttcacatgtgtggtgggaaggcttttacccaatgctctgtctttccagcccatcctcccttgttaactgccaaacagctgcctatcctgtccatgtgtagctcactgctacttcttttattatgaggtcagcacatgttactaaagatggcaagagaagaaggttctttcattgtgtcatagctatagctcaggaggaattttatttcctgtgtaggcacacaggagagcatcttccagctcacactccaactgaactaactgaacacctgcctatatatccaaagaaggggtgtcagtgccaatcacagcacacctccagtgcaaatgaaggtttgtgtttgcaccaatcacagccttgcctcttttagcatgcatcacaacaaagtcctcctagactatcaggggatatgctctcttggccaaggtaggaatagttgcagtgtcatctggcacaaaccatttcaaacggcctggctgaggttatgccttcgggaacctgaagtctttgtgtggttgtctccaagtgtctgtggagctccaggcggctggtgctgacagacgctttgtctagttggctgtttgacttttgcttaagcagccagggcagtagagtctaacagatgctaatttcaggatcaggaagactgtagaaaaatgagcatcaagaagcccctggtacccaaagctgctcttgccaatgagtgaacctctgccttcccgcttccaggtcctgtcttgaagaagaggttctggatcccgtgaatccgttcgtgcagctgctgagcggagtcgtgtggctcatccgcaatggaaacatctacatcaacgagagccaagccatcgagtgtgacgagacacaggagacaggtcaggaagcacaggtgttctgttttatttgtattaggttttgatttgtttattttgtgcatgcagcgggtgcatgcatgctcctttcctttcgccatgtgagtcctgagtattgaactcagactgttaagtgtgatgggaggcactttacccactgagccactttcccagccctcagcatcagctttcttcagacccaggaacagtgtgagtgggttattctttagtgttcccaaacatttactgagcagctatttactgtttagcactatggtgagagtcctagggattcagtcttatgtagaatatagaaggagaatccttggcaataagctggaaaattgtgacaagtgccaagaaagaaacaggagaaaggggaccggtggggaccagaagcacaggtatgaggaaagtgcctgcagatttgctgtatggtggcctccacatggcctaggagtttgtcataaatgcagagccatgagtccaccctccctatacctcccatccagaaaccactggttaaatcctaacaacttgggtgtgcaggcactcccttggtgactctgatggacactcaaggtcaagggccacttggggatgggctgatgagttggcttggtcagtaaagtatttgccttgaaagtgtgaggacctgagttggagccccagaaagaaacattaaaagccaagtgctgggatgcacacttgcattcccagggatggagctggaaggcagggataggcagatccacggccacacggtgatattctaagctaacaagagacctgtctcacacagaaagtgggtggcacctgaggaccaacacccagggttatcctctgacgtacctccagagtggaaaatactggggtggtggaaaaggacactttggtcctgggaatctggctattcagggtatagtgtagagggagagggagactcaagaggctgtctttgagtcaaaggaacaagctatcagaagaactcagggcagaggcctgtggttcccaggctcagggcagccttcaaggccctaggcagagagtagctgctgggtgaacaagtacagaagtgaggcctggggcctcaggcaaggcctgtgaaatccttccaccaacatagaagtttctggagactgagatcacatgaagtgcttctggctgtggcatggaagctcactggaggtggagctgggatgtggctcagtgatccagtgcttgccacacgtgcacgagggaaggagccatcaaaagagagaaagtcgggagacctgaggggtcccctggagagctgggtaaccaccccgggcccttctcctttaggttcttttagcaaatttgtgaatgtgatgtcagccaggacagccgtgggtcatgaccgtgaggggcagcttatcctcttccatgctgatggacagacggaacagcgtggtgagtcccaggaaccttggggctgtttgcacttcagccaccctacctttccagtcggttctggggtattggtgggacaagacagctttccggccattttggaagtttcatctggaggcaatagcatttacctactagtgaaagaagccagttaagccagagaccacaggggctcaagctgcataccccctctgcacagccttaacctatgggagatggcagagttcctgcgtcaacaagatgtcgtcaatgccatcaacctggatggaggcggttctgctacttttgtgctcaatgggaccctggccagttacccttcagatcactggtaagaacccttgagccacctttgtggctctctcagactgtctcactcagtcaatactgagaccctgttgtgtgccaggccctgggtatccaaaagtgagcagaagagccgagatctcttccctcagggtgctgcacagcccatccctggaaacctgagacaggtcaggaaaggcctccctgaggacagtgaagtaagacctgaggagatggctggccggggttgagagagcctttaccggaagacaaactgtacgcaatggggaaatccgctaagtggcccagggagaggctggagctatagctcaggaggaaaagtacttgcctcgcaagcgaaggacctgagtttaaactccaaaacccatataaaaagccagatacgagcaagtggcacatgcttgcagtcccagccttgttgaggaagagtcaggtgaatcctgaccctctggccagccagcctagcctactttttggcaaggtccaggccagcgagaaagataaataaaataaagttttaaatgacatgtatctaaggttgtcctgactccatatgcgcacgcacgcatgcacgcacgcacaactggcagaatggaaagggaggcaaactggacagcctttataggctgcggcagggaccagcaccaaggcctagacctcgtctcacagtgaatcccccacagccaggacaacatgtggcgctgtccccgccaagtgtccactgtggtgtgtgtgcatgaaccgcgctgccagccacccgactgcagtggccatgggacctgtgtggatggccactgtgaatgcaccagccacttctggcggggcgaggcctgcagcgagctggactgtggcccctccaactgcagccagcatgggctgtgcacagagagtgagtggggagcccacaggagggtggtgctctggcgggaccccagctcgcccatgctagactcccgcctgtgtccttacccagcctctgtggtcttgctttggtagctggctgccactgtgatgctgggtggacaggatccaactgcagtgaaggtgagagctgcctgcaaacactcctggagagggtggcctggctgcacgcagctggtatgacgccttcgtccctccttctggcttggaacttaccttcagagccttttctcatttcgcatgtggatacccgatgttctacctactgaaagagcccacaagtaggaagccagattttcagtattgtcactcaactctaaggaccaatagcaaaaaaacaaagtggccacgcccctgagggagatccaccaaagtccttaactcctggaaagcagctcctggtgatcctaggcatgggtagggtggtttcagcatcagctcagtggagttcccattcataatttcttcatccttttaaggtcataagttctagagcccaccttaaatctaggcagtattcttggtgtttatctgagacaaagtcttatacagcccacgcagttctctaacttagtatgtaaccgagaatggcctcaagcaacctgcttcctcctttcaagcgctgggattataggcatagcaccaacttatagggtgctagaagtcaaacccagggccctatgtatatgcagcaagcactctagaaactggaacacagccctgtttgcagcccggttaccttggagggttgggtcccagggatctgagggcatctccttcagcatggccatgtgcacacccaggagccaggctgtctgtgacaggagaccatgccacccaaggtgagacctccctgccaccatctcctctccacagagtgtcctctgggctggtatgggccaggttgccagaggccctgccagtgtgagcaccagtgtttctgtgacccgcagactggcaactgcagcatctcccaaggtatgcggccttaaaggttcttgagctgggagcccttggggcaggtctggggtaggtggactctccccagcccttctttctggtgtcttgcagtgaggcagtgtctccagccaactgaggctacgccgagggcaggagagctggcctctttcaccaggtaagtgttttagcaggcactgagcccctatgtctcatccgtgaggcactagccaggccaggaggtcacaggttaccctctactttgcaagctcagggacagtcacaggtaaaactggcatccaggaaagaccctgagctacccagtggaactcaaaggtagcaggctatgggtgtcatgcctctggctgcagagactccacttagatgctggagcagggccatagagacaggaaggactcaccttatttctgaactcttccgtgtgttcaggctttgtgttgttgttgcttcctttctgctgtttcctgggtttccagctccatccccacagggctcatggaaagaattgtgaagcagggggtgtggctcaattggcagattgattgcctggcatgcagaaagccctaggttcaatccccagcatttcatatcataacccaggcatggtggcatcatgtgcctgtaagtccagcacttgggaggtagaagcagaaaagccacgagtttaagaatgttagggagtcttaggccaacctgggatacctaagacaagagatagatgtagggagatagattgacagacagacagacagacagacagacagacagatcttgagctggaccttctggcacaagcctgtcatcctagctattccaggaagctgaagcaggaagatagcaaattcaaggccagcttaagccacagattgagttcaagatcaacctgagcaactttatgaaatcctattataacataaaaagtaggggtgggaggttaggctgtagctcagtggtagagtgattgcctagcacgcacaagacccaggttcaattcccagtactgcaaaaaatatattaggaaccccctaaaagcagtaacattcacattagatgtgtgtgtgtgtgtgtgtgtgtgtgtgtgtgtgtgtgtgtgtgtgtgtgtgttttgttgggtatttatttcatttacatttccaatgctatcccaaaagtcccccacatcctcccccacccaccaccttgtttttttttttttttttttttttttttttgacctgaaactcacaggttaggttagacaagctgactggtgagctccaacttccaacgtaccatcatgcctggcttttgttttggtgtctctgtgtaaccctggatgtcctggagctctctctgtagaccagcctggccttaaactcacagaaacccacctgtttctgcctcccatgtgctgggattaaaggcgtgtgccacctcacccagccctgctggacttaaattgggtcttcattttataagacaagcatgagctaattccccagttcctaaaatgtttttaacatccttaaacatcagagactgtctgtggtattccctccatgtgtcttcagtatacctactcccctccctgcctactgggttcaacatgcccagtttgggttctggctgcctgcccccactcaagactctcttttccatctcaggaccacctggctagccctcaccctgacactaattttcctgctgctgatcagcactggggtcaacgtgtccttgttcctgggctccagggccgagaggaaccggcacctcgacggggactatgtgtatcacccactgcaggaggtgaacggggaagcgctgactgcagagaaggagcacatggaggaaactagcaaccccttcaaggactgaagagctgccccaacggcatgctccagataatcttgtccctgctcctcacttccacaggggacattgtgaggccactggcatggatgctatgcaccccaccctttgctggccatattcctcctgtccccatgctgtggctcatgccaacctagcaataaggagctctggagagcctgcacctgcctcccgctcgcctatatctgctgcccagaggcctgtctcgcacaggggtctcgccactgccaaagactcccaggaagtcaaagactcccagtaatccactagcaaatggaactctgtaacgccatcataacaagagtggccactctccgcgtgcacaggtatgaaatataaatccttacacacacacacacacacaccctcggctcagccacggcactcgccttttatacagcgtcatcgctggacagccaactagaactctgcatcctgtcacaggaagcacctcataagaaggaatggggagggaaggcagtcgccttgttttcagaccttagccgaattc

SEQ ID NO: 22 - protein, 20 aa, Artificial Sequence
  synthetic peptide
METDTLLLWVLLLWVPGSTG

SEQ ID NO: 23 - protein, 13 aa, Artificial Sequence
  synthetic peptide
DEDQVDPRLIDGK

SEQ ID NO: 24 - DNA, 2279 bp, Homo sapiens
agctaaggcaggtacctgcatccttgtttttgtttagtggatcctctatccttcagagactctggaacccctgtggtcttctcttcatctaatgaccctgaggggatggagttttcaagtccttccagagaggaatgtcccaagcctttgagtagggtaagcatcatggctggcagcctcacaggattgcttctacttcaggcagtgtcgtgggcatcaggtgcccgcccctgcatccctaaaagcttcggctacagctcggtggtgtgtgtctgcaatgccacatactgtgactcctttgaccccccgacctttcctgcccttggtaccttcagccgctatgagagtacacgcagtgggcgacggatggagctgagtatggggcccatccaggctaatcacacgggcacaggcctgctactgaccctgcagccagaacagaagttccagaaagtgaagggatttggaggggccatgacagatgctgctgctctcaacatccttgccctgtcaccccctgcccaaaatttgctacttaaatcgtacttctctgaagaaggaatcggatataacatcatccgggtacccatggccagctgtgacttctccatccgcacctacacctatgcagacacccctgatgatttccagttgcacaacttcagcctcccagaggaagataccaagctcaagatacccctgattcaccgagccctgcagttggcccagcgtcccgtttcactccttgccagcccctggacatcacccacttggctcaagaccaatggagcggtgaatgggaaggggtcactcaagggacagcccggagacatctaccaccagacctgggccagatactttgtgaagttcctggatgcctatgctgagcacaagttacagttctgggcagtgacagctgaaaatgagccttctgctgggctgttgagtggataccccttccagtgcctgggcttcacccctgaacatcagcgagacttcattgcccgtgacctaggtcctaccctcgccaacagtactcaccacaatgtccgcctactcatgctggatgaccaacgcttgctgctgccccactgggcaaaggtggtactgacagacccagaagcagctaaatatgttcatggcattgctgtacattggtacctggactttctggctccagccaaagccaccctaggggagacacaccgcctgttccccaacaccatgctctttgcctcagaggcctgtgtgggctccaagttctgggagcagagtgtgcggctaggctcctgggatcgagggatgcagtacagccacagcatcatcacgaacctcctgtaccatgtggtcggctggaccgactggaaccttgccctgaaccccgaaggaggacccaattgggtgcgtaactttgtcgacagtcccatcattgtagacatcaccaaggacacgttttacaaacagcccatgttctaccaccttggccacttcagcaagttcattcctgagggctcccagagagtggggctggttgccagtcagaagaacgacctggacgcagtggcactgatgcatcccgatggctctgctgttgtggtcgtgctaaaccgctcctctaaggatgtgcctcttaccatcaaggatcctgctgtgggcttcctggagacaatctcacctggctactccattcacacctacctgtggcgtcgccagtgatggagcagatactcaaggaggcactgggctcagcctgggcattaaagggacagagtcagctcacacgctgtctgtgactaaagagggcacagcagggccagtgtgagcttacagcgacgtaagcccaggggcaatggtttgggtgactcactttcccctctaggtggtgccaggggctggaggcccctagaaaaagatcagtaagccccagtgtccccccagcccccatgcttatgtgaacatgcgctgtgtgctgcttgctttggaaactgggcctgggtccaggcctagggtgagctcactgtccgtacaaacacaagatcagggctgagggtaaggaaaagaagagactaggaaagctgggcccaaaactggagactgtttgtctttcctggagatgcagaactgggcccgtggagcagcagtgtcagcatcagggcggaagccttaaagcagcagcgggtgtgcccaggcacccagatgattcctatggcaccagccaggaaaaatggcagctcttaaaggagaaaatgtttgagcccaaaaaaaaaaaaaaaaaaaaa

SEQ ID NO: 25 - protein, 536 aa, Homo sapiens
MEFSSPSREECPKPLSRVSIMAGSLTGLLLLQAVSWASGARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWRRQ

SEQ ID NO: 26 - protein, 536 aa, Homo sapiens
MEFSSPSREECPKPLSRVSIMAGSLTGLLLLQAVSWASGARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWHRQ

SEQ ID NO: 27 - protein, 6 aa, Artificial Sequence
  synthetic peptide
RARYKR

(19) United States
(12) Patent Application Publication
(10) Pub. No.: US 2003/0133924 A1
(43) Pub. Date: Jul. 17, 2003

(54) Highly phosphorylated acid beta-glucocerebrosidase and methods of treating gaucher's disease

(75) Inventor: William Canfield, Oklahoma City, OK (US)

Correspondence Address: OBLON, SPIVAK, MCCLELLAND, MAIER & NEUSTADT, P.C., 1940 DUKE STREET, ALEXANDRIA, VA, 22314, US

(73) Assignee: NOVAZYME PHARMACEUTICALS, INC., Oklahoma City, OK

(21) Appl. No.: 10/024,197

(22) Filed: Dec. 21, 2001

(51) Int. Cl. A61K 38/47; C07H 21/04; C12P 21/06; C12N 9/36; C12P 21/02; C12N 5/06

(52) U.S. Cl. 424/94.61; 435/68.1; 435/206; 435/69.1; 435/320.1; 435/325; 536/23.2

(57) ABSTRACT

The present invention provides a highly phosphorylated acid beta-glucocerebrosidase (GBA), which can be employed in an enzyme replacement therapy protocol to treat patients suffering from Gaucher's disease.

### BACKGROUND OF THE INVENTION

1. Field of the Invention

The present invention is directed to a highly phosphorylated acid beta-glucocerebrosidase (GBA), which can be employed in an enzyme replacement therapy protocol to treat patients suffering from Gaucher's disease.

2. Discussion of the Background

Gaucher's disease is a lysosomal storage disease believed to be caused by a deficiency of acid β-glucocerbrosidase (GBA)(Friedman, B., et al (1999) Blood, 93, 2807-2816). Most lysosomal enzymes are targeted to the lysosome by the mannose 6-phosphate (M6P) dependent pathway. In order for lysosomal enzymes to be targeted to the lysosome they must first acquire, through post-translational modification the M6P residues essential for targeting. GBA is an exception in that it is not believed to be naturally targeted through the M6P pathway or is it completely understood how it is targeted.

These post-translational modifications may be carried out by the sequential action of two enzymes: N-acetylglucosaminylphosphotransferase (GlcNAc-phosphotransferase) and N-acetylglucosamine-1-phosphodiester α-N-acetylglucosaminidase (Uncovering enzyme; UCE) (Varki, A. P., et al (1981) Proc. Natl. Acad. Sci. USA., 78, 7773-7777).

GlcNAc-phosphotransferase catalyzes the transfer of N-acetylglucosamine-1-phosphate from UDP-GlcNAc to the 6 position of 1,2-linked mannoses on the lysosomal enzyme. The recognition and addition of N-acetylgluocosamine-1-phosphate to lysosomal hydrolases by GlcNAc-phosphotransferase is the critical and determining step in lysosomal targeting. The second step is catalyzed by N-acetylglucosamine-1-phosphodiester α-N-Acetylglucosaminidase (“phosphodiester α-GlcNAcase”) (E.C. 3.1.4.45). Phosphodiester α-GlcNAcase catalyzes the removal of N-Acetylglucosamine from the GlcNAc-phosphate modified lysosomal enzyme to generate a terminal M6P on the lysosomal enzyme.

Both enzymes responsible for the terminal M6P on the lysosomal enzymes have been previously isolated and characterized (U.S. Pat. Ser. Nos. 09/636,077, 09/636,596, 09/635,872 and 09/636,060, incorporated herein by reference). During normal cellular processes, GBA is not typically a substrate for the GlcNAc phosphotransferase/phosphodiester α-GlcNAcase modification pathway.

Currently, the GBA used in enzyme replacement therapy is modified so that it contains terminal mannose moieties (2 GlcNAc and 3 mannose) that facilitate GBA targeting to tissues via the high affinity mannose receptor located on the surface of some macrophages (Friedman et (1999) Blood:93(9):2807-2816). A problem that exist with the current GBA enzyme replacement therapy is that affected tissues such as bone and lung in which the enzyme is unable to reach because these tissues do not contain the proper macrophages to allow efficient targeting (Beutler, E. et al (1995) Mol Med, 1, 320-324). Thus, while the tissues that are targeted by the current GBA, e.g., liver and spleen, receive some benefit from the replacement therapy, the tissues that are not targeted, e.g., bone and lung, suffer from long-term deficiencies such as pulmonary hypertension and progressive bone disease (Gaucher's bone disease) (see, for example, Beutler et al (1995) Mol. Med., 1:320-324).

To address these problems with current GBA replacement therapy, GBA will be phosphorylated which will allow binding to mannose 6 receptors on the surface of lung and bone cells. In so binding to the receptor on these tissues the problems of the current GBA replacement therapy can be addressed. Therefore, the highly phosphorylated GBA when employed in therapeutic protocols will increase the amount of GBA in the targeted bone and lung tissues resulting in improvements for the long-term prospects of Gaucher's patients. (Friedman et al (1999) Blood:93(9):2807-2816).

The present inventors have discovered that GlcNAc phosphotransferase, comprising the α and β subunits reduces substrate specificity, which allows the GlcNAc phosphotransferase to catalyze the transfer of N-acetylglucosamine-1-phosphate from UDP-GlcNAc to the GBA enzyme. This modified GBA may then be treated with phosphodiester α-GlcNAcase to complete the modification of the GBA thereby making the enzyme available for targeting tissues via the M6P receptor.

This modified enzyme is found to bind to the mannose 6-phosphate receptor with high affinity resulting in an increased bioavailablity of the enzyme to mannose 6-phosphate bearing cells when compared to the current GBA employed in therapeutic protocols.

### SUMMARY OF THE INVENTION

Accordingly, one object of the present invention is a method of preparing a highly phosphorylated acid β-glucocerbrosidase comprising contacting said acid β-glucocerbrosidase with an isolated GlcNAc phosphotransferase to produce a modified acid β-glucocerbrosidase; and contacting said modified acid β-glucocerbrosidase with an isolated phosphodiester α-GlcNAcase. In a preferred embodiment the highly phosphorylated acid β-glucocerbrosidase is purified after modification with the isolated GlcNAc phosphotransferase or after contacting with the isolated phosphodiester α-GlcNAcase.

Another object of the present invention is a method of preparing the highly phosphorylated GBA by culturing transfected cells comprising a recombinant polynucleotide which encodes a recombinant acid β-glucocerbrosidase in the presence of at least one α 1,2-mannosidase inhibitor; recovering a high mannose recombinant acid β-glucocerbrosidase from said transfected cell; contacting said high mannose recombinant acid β-glucocerbrosidase with an isolated GlcNAc phosphotransferase to produce a modified acid β-glucocerbrosidase; and contacting said modified acid β-glucocerbrosidase with an isolated phosphodiester α-GlcNAcase.

Another object of the present invention is a highly phosphorylated acid β-glucocerbrosidase.

Another object of the present invention pharmaceutical compositions that contain highly phosphorylated acid β-glucocerbrosidase with a pharmaceutically acceptable carrier.

Another object of the present invention is a method of treating a patient suffering from Gaucher's disease by administering the highly phosphorylated acid β-glucocerbrosidase.

In one embodiment, the method entails administration to bone and/or lung tissue of the patient. In another embodiment, the highly phosphorylated acid β-glucocerbrosidase is administered with a acid β-glucocerbrosidase which is not highly phosphorylated.

### BRIEF DESCRIPTION OF THE DRAWING

FIG. 1: Mannose-6-Phosphate column comparison of (A) wildtype GBA; (B) highly phosphorylated GBA, phosphorylated with a mixture of α/β/γ GlcNAc-phosphotransferase and α/β GlcNAc-phosphotransferase; and (C) highly phosphorylated GBA, phosphorylated with α/β GlcNAc-phosphotransferase.

### DETAILED DESCRIPTION OF THE INVENTION

Unless otherwise defined, all technical and scientific terms used herein have the same meaning as commonly understood by one of ordinary skill in the art of molecular biology. Although methods and materials similar or equivalent to those described herein can be used in the practice or testing of the present invention, suitable methods and materials are described herein. All publications, patent applications, patents, and other references mentioned herein are incorporated by reference in their entirety. In addition, the materials, methods, and examples are illustrative only and are not intended to be limiting.

Reference is made to standard textbooks of molecular biology that contain definitions and methods and means for carrying out basic techniques, encompassed by the present invention. See, for example, Sambrook et al., Molecular Cloning: A Laboratory Manual, Third Edition, Cold Spring Harbor Laboratory Press, New York (2001), Current Protocols in Molecular Biology, Ausebel et al (eds.), John Wiley & Sons, New York (2001) and the various references cited therein.

“Isolated” means separated out of its natural environment.

“Polynucleotide” in general relates to polyribonucleotides and polydeoxyribonucleotides, it being possible for these to be non-modified RNA or DNA or modified RNA or DNA.

“Polypeptides” are understood as meaning peptides or proteins which comprise two or more amino acids bonded via peptide bonds.

The term “acid β-glucocerbrosidase” or “GBA” as used herein refers to enzymes that are involved in glycolipid degradation while in Gaucher deficiency in all cells and tissue, the predominant problem is in reticuloendothelial cells. GBA is also known in the art as acid β-glucosidase.

Polynucleotides which encode GBA as used herein is understood to mean the sequences exemplified in this application as well as those which have substantial identity to SEQ ID NO: 24 (shown below) and which encode an enzyme having GBA activity. The cDNA comprising SEQ ID NO:24 can produce type I and type II GBA. Initiation codons and stop codon are shown in bold. In one preferred embodiment for the polynucleotide sequence expression a Kozak sequence can be introduced upstream of the initiation codon for better expression and the 3′-UTR can be deleted.

agctaaggca ggtacctgca tccttgtttt tgtttagtgg
atcctctatc cttcagagac tctggaaccc ctgtggtctt
ctcttcatct aatgaccctg aggggatgga gttttcaagt
ccttccagag aggaatgtcc caagcctttg agtagggtaa
gcatcatggc tggcagcctc acagqattgc ttctacttca
ggcagtgtcg tgggcatcag gtgcccgccc ctgcatccct
aaaagcttcg gctacagctc ggtggtgtgt gtctgcaatg
ccacatactg tgactccttt gaccccccga cctttcctgc
ccttggtacc ttcagccgct atgagagtac acgcagtggg
cgacggatgg agctgagtat ggggcccatc caggctaatc
acacgggcac aggcctgcta ctgaccctgc agccagaaca
gaagttccag aaagtgaagg gatttggagg ggccatgaca
gatgctgctg ctctcaacat ccttgccctg tcaccccctg
cccaaaattt gctacttaaa tcgtacttct ctgaagaagg
aatcggatat aacatcatcc gggtacccat ggccagctgt
gacttctcca tccgcaccta cacctatgca gacacccctg
atgatttcca gttgcacaac ttcagcctcc cagaggaaga
taccaagctc aagatacccc tgattcaccg agccctgcag
ttggcccagc gtcccgtttc actccttgcc agcccctgga
catcacccac ttggctcaag accaatggag cggtgaatgg
gaaggggtca ctcaagggac agcccggaga catctaccac
cagacctggg ccagatactt tgtgaagttc ctggatgcct
atgctgagca caagttacag ttctgggcag tgacagctga
aaatgagcct tctgctgggc tgttgagtgg ataccccttc
cagtgcctgg gcttcacccc tgaacatcag cgagacttca
ttgcccgtga cctaggtcct accctcgcca acagtactca
ccacaatgtc cgcctactca tgctggatga ccaacgcttg
ctgctgcccc actgggcaaa ggtggtactg acagacccag
aagcagctaa atatgttcat ggcattgctg tacattggta
cctggacttt ctggctccag ccaaagccac cctaggggag
acacaccgcc tgttccccaa caccatgctc tttgcctcag
aggcctgtgt gggctccaag ttctgggagc agagtgtgcg
gctaggctcc tgggatcgag ggatgcagta cagccacagc
atcatcacga acctcctgta ccatgtggtc ggctggaccg
actggaacct tgccctgaac cccgaaggag gacccaattg
ggtgcgtaac tttgtcgaca gtcccatcat tgtagacatc
accaaggaca cgttttacaa acagcccatg ttctaccacc
ttggccactt cagcaagttc attcctgagg gctcccagag
agtggggctg gttgccagtc agaagaacga cctggacgca
gtggcactga tgcatcccga tggctctgct gttgtggtcg
tgctaaaccg ctcctctaag gatgtgcctc ttaccatcaa
ggatcctgct gtgggcttcc tggagacaat ctcacctggc
tactccattc acacctacct gtggcgtcgc cagtgatgga
gcagatactc aaggaggcac tgggctcagc ctgggcatta
aagggacaga gtcagctcac acgctgtctg tgactaaaga
gggcacagca gggccagtgt gagcttacag cgacgtaagc
ccaggggcaa tggtttgggt gactcacttt cccctctagg
tggtgccagg ggctggaggc ccctagaaaa agatcagtaa
gccccagtgt ccccccagcc cccatgctta tgtgaacatg
cgctgtgtgc tgcttgcttt ggaaactggg cctgggtcca
ggcctagggt gagctcactg tccgtacaaa cacaagatca
gggctgaggg taaggaaaag aagagactag gaaagctggg
cccaaaactg gagactgttt gtctttcctg gagatgcaga
actgggcccg tggagcagca gtgtcagcat cagggcggaa
gccttaaagc agcagcgggt gtgcccaggc acccagatga
ttcctatggc accagccagg aaaaatggca gctcttaaag
gagaaaatgt ttgagcccaa aaaaaaaaaa aaaaaaaaa

Preferably, polynucleotides that encode GBA are those which hybridize under stringent conditions and are at least 70%, preferably at least 80% and more preferably at least 90% to 95% identical to SEQ ID NO:24. GBA polynucleotides as herein also include those nucleotide sequences found in public databases, for example, those listed below (the corresponding protein ID is shown in parentheses):

| | | |
|---|---|---|
| 1. | BC 003356 | (AAH 03356.1) |
| 2. | D 13286 | (BAA 02545) |
| 3. | M 19285 | (AAA 35880) |
| 4. | M 16328 | (AAA 35873) |
| 5. | K 02920 | (AAA 35887) |
| 6. | BC 000349 | |
| 7. | J 03059 | (AAC 63056) |

| | |
|---|---|
| 8. | BG 716343 (full length with mutation); and |
| 9. | BG 281198 (not full length) |

The GBA protein or polypeptide as used herein is understood to mean the sequences exemplified in this application as well as those which have substantial identity to SEQ ID NO:25 and/or 26. Preferably, such polypeptides are those which are at least 70%, preferably at least 80% and more preferably at least 90% to 95% identical to SEQ ID NO:25 and/or 26. For example, the precursor protein of acid beta glucosidase (GBA, also known as glucocerebrosidase), include four different types of precursor protein. The amino acid sequence of this protein is depicted below (SEQ ID NO:25):

MEFSSPSREE CPKPLSRVSI MAGSLTGLLL LQAVSWASGA
RPCIPKSFGY SSVVCVCNAT YCDSFDPPTF PALGTFSRYE
STRSGRRMEL SMGPIQANHT GTGLLLTLQP EQKFQKVKGF
GGAMTDAAAL NILALSPPAQ NLLLKSYFSE EGIGYNIIRV
PMASCDFSIR TYTYADTPDD FQLHNFSLPE EDTKLKIPLI
HRALQLAQRP VSLLASPWTS PTWLKTNGAV NGKGSLKGQP
GDIYHQTWAR YFVKFLDAYA EHKLQFWAVT AENEPSAGLL
SGYPFQCLGF TPEHQRDFIA RDLGPTLANS THHNVRLLML
DDQRLLLPHW AKVVLTDPEA AKYVHGIAVH WYLDFLAPAK
ATLGETHRLF PNTMLFASEA CVGSKFWEQS VRLGSWDRGM
QYSHSIITNL LYHVVGWTDW NLALNPEGGP NWVRNFVDSP
IIVDITKDTF YKQPMFYHLG HFSKFIPEGS QRVGLVASQK
NDLDAVALMH PDGSAVVVVL NRSSKDVPLT IKDPAVGFLE
TISPGYSIHT YLWRRQ*
HRQ

From this amino acid sequence, the four different types include:

| | |
|---|---|
| Protein type I; | N-terminal is Met 1 and C-terminal is RRQ. |
| | (SEQ ID NO:25) |
| Protein type II; | N-terminal is Met 21 and C-terminal is RRQ. |
| Protein type III; | N-terminal is Met 1 and C-terminal is HRQ. |
| | (SEQ ID NO:26) |
| Protein type IV; | N-terminal is Met 21 and C-terminal is HRQ. |

In addition to the sequence depicted above, other suitable GBA sequences are known in the art as shown in the Table below, where several GBA proteins are identified by protein ID, reference number, or locus names.

| Type I | Type II | Type II | Type IV |
|---|---|---|---|
| CAD 12720 | AAA 35877 | NP 000148 | CAD 12721 |
| T 08828 | 1202301 A | P 04062 | AAA 35873 |
| BAA 02545 | EUHUGC | | |
| AAC 63056 | AAA 35880 | | |
| AAC 51820 | 1112264A | | |
| 2004300A | | | |
| AAH 03356.1 | | | |

The term “GlcNAc-phosphotransferase” as used herein refers to enzymes that are capable of catalyzing the transfer of N-acetylglucosamine-1-phosphate from UDP-GlcNAc to the 6′ position of 1,2-linked mannoses on lysosomal enzymes. The GlcNAc-phosphotrasferase is composed of six subunits: 2 α subunits, 2 β-subunits and 2 γ subunits. The amino acid sequence of the a subunit is shown in SEQ ID NO:4 (amino acids 1-928), the human β subunit is shown in SEQ ID NO:5 (amino acids 1-328), and the human γ subunit is shown in SEQ ID NO:7 (amino acids 25-305, signal sequence is in amino acids 1-24).

A novel soluble GlcNAc phosphotransferase has been prepared which is composed of a non-endogenous proteolytic cleavage site interposed between the α and β subunits. When combined with the γ subunit, this GlcNAc phosphotransferase exhibits high levels of activity. The soluble GlcNAc-phosphotransferase protein or polypeptide as used herein is understood to mean the sequences exemplified in this application as well as those which have substantial identity to SEQ ID NO:2. The partial rat and Drosphila melanogaster α/β GlcNAc-phosphotransferase amino acid sequences are shown in SEQ ID NO: 14 and 16, respectively.

Preferably, the GlcNAc-phosphotransferase polypeptides are those which are at least 70%, preferably at least 80% and more preferably at least 90% to 95% identical to the GlcNAc-phosphotransferase amino acid sequences described herein.

Polynucleotides which encode the α and β subunits of GlcNAc-phosphotransferase or soluble GlcNAc-phosphotransferase mean the sequences exemplified in this application as well as those which have substantial identity to those sequences and which encode an enzyme having the activity of the α and β subunits of GlcNAc-phosphotransferase. Preferably, such polynucleotides are those which hybridize under stringent conditions and are at least 70%, preferably at least 80% and more preferably at least 90% to 95% identical to those sequences

The nucleotide sequence for the human α/β subunit precursor cDNA is shown in SEQ ID NO:3 (nucleotides 165-3932), the nucleotide sequence of the α subunit is in nucleotides 165-2948 of SEQ ID NO:3, the nucleotide sequence of the β subunit is shown in nucleotides 2949-3932 of SEQ ID NO:3, and the nucleotide sequence of the γ subunit is shown in SEQ ID NO:6 (nucleotides 24-95). The soluble GlcNAc-phosphotransferase nucleotide sequence is shown in SEQ ID NO:1. The partial rat and Drosphila melanogaster α/β GlcNAc-phosphotransferase nucleotide sequences are shown in SEQ ID NO: 13 and 15, respectively.

The term “phosphodiester α-GlcNAcase” as used herein refers to enzymes that are capable of catalyzing the removal of N-Acetylglucosamine from GlcNAc-phosphate-mannose diester modified lysosomal enzymes to generate terminal M6P.

Polynucleotides which encode phosphodiester α-GlcNAcase as used herein is understood to mean the sequences exemplified in this application as well as those which have substantial identity to SEQ ID NO:19 (murine) or SEQ ID NO:17 (human) and which encode an enzyme having the activity of phosphodiester α-GlcNAcase. Preferably, such polynucleotides are those which hybridize under stringent conditions and are at least 70%, preferably at least 80% and more preferably at least 90% to 95% identical to SEQ ID NOS:17 and/or 19.

The phosphodiester α-GlcNAcase protein or polypeptide as used herein is understood to mean the sequences exemplified in this application as well as those which have substantial identity to SEQ ID NO:20 (murine) or SEQ ID NO:18 (human). Preferably, such polypeptides are those which are at least 70%, preferably at least 80% and more preferably at least 90% to 95% identical to SEQ ID NOS:18 and/or 20.

The terms “stringent conditions” or “stringent hybridization conditions” includes reference to conditions under which a polynucleotide will hybridize to its target sequence, to a detectably greater degree than other sequences (e.g., at least 2-fold over background). Stringent conditions are sequence-dependent and will be different in different circumstances. By controlling the stringency of the hybridization and/or washing conditions, target sequences can be identified which are 100% complementary to the probe (homologous probing). Alternatively, stringency conditions can be adjusted to allow some mismatching in sequences so that lower degrees of similarity are detected (heterologous probing).

Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. Exemplary low stringency conditions include hybridization with a buffer solution of 30 to 35% formamide, 1 M NaCl, 1% SDS (sodium dodecyl sulphate) at 37° C., and a wash in 1× to 2×SSC (20×SSC=3.0 M NaCl/0.3 M trisodium citrate) at 50 to 55° C. Exemplary moderate stringency conditions include hybridization in 40 to 45% formamide, 1 M NaCl, 1% SDS at 37° C., and a wash in 0.5× to 1×SSC at 55 to 60° C. Exemplary high stringency conditions include hybridization in 50% formamide, 1 M NaCl, 1% SDS at 37° C., and a wash in 0.1×SSC at 60 to 65° C.

Specificity is typically the function of post-hybridization washes, the critical factors being the ionic strength and temperature of the final wash solution. For DNA-DNA hybrids, the Tₘ can be approximated from the equation of Meinkoth and Wahl, Anal. Biochem., 138:267-284 (1984): Tₘ=81.5° C.+16.6 (log M)+0.41 (% GC)−0.61 (% form)−500/L; where M is the molarity of monovalent cations, % GC is the percentage of guanosine and cytosine nucleotides in the DNA, % form is the percentage of formamide in the hybridization solution, and L is the length of the hybrid in base pairs. The Tₘ is the temperature (under defined ionic strength and pH) at which 50% of a complementary target sequence hybridizes to a perfectly matched probe. Tₘ is reduced by about 1° C. for each 1% of mismatching; thus, Tₘ, hybridization and/or wash conditions can be adjusted to hybridize to sequences of the desired identity. For example, if sequences with approximately 90% identity are sought, the Tₘ can be decreased 10° C. Generally, stringent conditions are selected to be about 5° C. lower than the thermal melting point (Tₘ) for the specific sequence and its complement at a defined ionic strength and pH. However, severely stringent conditions can utilize a hybridization and/or wash at 1, 2, 3, or 4° C. lower than the thermal melting point (Tₘ); moderately stringent conditions can utilize a hybridization and/or wash at 6, 7, 8, 9, or 10° C. lower than the thermal melting point (Tₘ); low stringency conditions can utilize a hybridization and/or wash at 11, 12, 13, 14, 15, or 20° C. lower than the thermal melting point (Tₘ). Using the equation, hybridization and wash compositions, and desired Tₘ, those of ordinary skill will understand that variations in the stringency of hybridization and/or wash solutions are inherently described. If the desired degree of mismatching results in a Tₘ of less than 45° C. (aqueous solution) or 32° C. (formamide solution) it is preferred to increase the SSC concentration so that a higher temperature can be used. An extensive guide to the hybridization of nucleic acids is found in Tijssen, Laboratory Techniques in Biochemistry and Molecular Biology—Hybridization with Nucleic Acid Probes, Part I, Chapter 2 “Overview of principles of hybridization and the strategy of nucleic acid probe assays”, Elsevier, N.Y. (1993); and Current Protocols in Molecular Biology, Chapter 2, Ausubel, et al., Eds., Greene Publishing and Wiley-Interscience, New York (1995).

Homology, sequence similarity or sequence identity of nucleotide or amino acid sequences may be determined conventionally by using known software or computer programs such as the BestFit or Gap pairwise comparison programs (GCG Wisconsin Package, Genetics Computer Group, 575 Science Drive, Madison, Wis. 53711). BestFit uses the local homology algorithm of Smith and Waterman, Advances in Applied Mathematics 2: 482-489 (1981), to find the best segment of identity or similarity between two sequences. Gap performs global alignments: all of one sequence with all of another similar sequence using the method of Needleman and Wunsch, J. Mol. Biol. 48:443-453 (1970). When using a sequence alignment program such as BestFit, to determine the degree of sequence homology, similarity or identity, the default setting may be used, or an appropriate scoring matrix may be selected to optimize identity, similarity or homology scores. Similarly, when using a program such as BestFit to determine sequence identity, similarity or homology between two different amino acid sequences, the default settings may be used, or an appropriate scoring matrix, such as blosum45 or blosum80, may be selected to optimize identity, similarity or homology scores.

The high-affinity ligand for the cation-independent M6P receptor is an oligosaccharide containing two M6P groups (i.e., a bis-phosphorylated oligosaccharide). Since a bis-phosphorylated oligosaccharides binds with an affinity 3500-fold higher than a monophosphorylated oligosaccharides, virtually all the high-affinity binding of a lysosomal enzyme to the M6P receptor will result from the content of bis-phosphorylated oligosaccharides (Tong, P. Y., Gregory, W., and Kornfeld, S. (1989)). “Ligand interactions of the cation-independent mannose 6-phosphate receptor. The stoichiometry of mannose 6-phosphate binding.” Journal of Biological Chemistry 264: 7962-7969). It is therefore appropriate to use the content of bis-phosphorylated oligosaccharides to compare the binding potential of different preparations of GBA.

The phrase “highly phosphorylated GBA” as used herein refers to GBA which contains more bis-phosphorylated oligosaccharides compared to known naturally occurring or recombinant GBA. Preferably, GBA contains at least 5% bis-phosphorylated oligosaccharides compared to GBA not treated with the GlcNAc-phosphotransferase described herein. More preferably, the “highly phosphorylated GBA” has at least 6%, 7%, 8%, 9%, 10%, 11%, 12%, 13%,14%, 15%, 16%, 17%, 18%, 19%, 20%, 21%, 22%,23%, 24%, 25%, 26%, 27%, 28%, 29%,30%, 40%,45%, 50%, 60%, 70%, 80%, 85%, 90%, 95%, 100% bis-phosphorylated oligosaccharides and all values and ranges there between. This highly phosphorylated GBA have a higher affinity for the M6P receptor and are therefore more efficiently taken into the cell by plasma membrane receptors.

The phrase “highly phosphorylated GBA” as used herein refers to GBA which is more highly phosphorylated naturally or recombinant GBA. Preferably, highly phosphorylated GBA contains at least 5% of the molecules that bind with high affinity to a mannose 6 phosphate column. More preferably, the “highly phosphorylated GBA” has at least 6%, 7%, 8%, 9%, 10%, 11%, 12%, 13%,14%, 15%, 16%, 17%, 18%, 19%, 20%, 21%, 22%,23%, 24%, 25%, 26%, 27%, 28%, 29%,30%, 40%,45%, 50%, 60%, 70%, 80%, 85%, 90%, 95%, 100% mannose 6 receptor binding high affinity GBA and all values and ranges there between. This highly phosphorylated GBA have a higher affinity for the M6P receptor and are therefore more efficiently taken into the cell by plasma membrane receptors.

The high-affinity ligand for the cation-independent M6P receptor is an oligosaccharide containing two M6P groups (i.e., a bis-phosphorylated oligosaccharide). Since a bisphosphorylated oligosaccharides binds with an affinity 3500-fold higher than a monophosphorylated oligosaccharides, virtually all the high-affinity binding of a lysosomal enzyme to the M6P receptor will result from the content of bis-phosphorylated oligosaccharides (Tong, P. Y., Gregory, W., and Kornfeld, S. (1989)). “Ligand interactions of the cation-independent mannose 6-phosphate receptor. The stoichiometry of mannose 6-phosphate binding.” Journal of Biological Chemistry 264: 7962-7969). It is therefore appropriate to use the content of bis-phosphorylated oligosaccharides to compare the binding potential of different preparations of lysosomal enzymes.

In addition to measuring the highly phosphorylated GBA using the M6P binding assay described herein, the extent of phosphorylation and thus, uptake of the highly phosphorylated GBA can be measured using a fibroblast uptake protocol.

This fibroblast uptake protocol may be conducted as follows:

Enzyme preparation: Dilute an enzyme preparation in PBS (pH 7.2) and plate in triplicate into a black 96well plate (25 μl/well). The amount of purified enzyme is equivalent to 1 million counts, this is the amount added per well to the cells in the uptake assay.

Uptake assay. Using a confluent flask of fibroblasts (for example, GM00372, GM 04394 and GM 07968, which are Gaucher disease type I and GM01260, and GM 00877 Gaucher disease type II, and GM 10915 Gaucher disease, type uncertain, all are accession numbers at the Coriel Cell Repository, 401 Haddon Avenue, Camden, N.J. 08103) aspirate the medium and wash cells in 10 ml of DPBS, aspirate DPBS, add 3 ml of trypsin to the flask and rock to coat the cells, incubate for approximately 5 minutes. Resuspend in 7 ml Dulbeccos' modified essential media (DMEM), final volume should be 10 ml. Count the number of cells in a hemacytometer and dilute suspension to produce 150,000 cells/ml using DMEM, plate 3 ml of cell suspension into four 60 mm culture dishes and incubate at 37° C. overnight. The next day change the medium on each dish to uptake medium (containing Ham's F-12, 10% Heat-Inactivated FBS, 3 mM Pipes, pH 6.7). Two hours later add 15 μl mannose-6-phosphate to each of two dishes. Two hours later 1 million fluorescent counts of the enzyme is added to the one of the two dishes having mannose-6-phosphate and the other to a dish containing only PBS (if the enzymatic activity is a fluorescent assay, e.g., using 4-MU-β-glucose, as described herein, it is preferable to employ a dark or black plate; and if a colorometric assay, e.g., using BCA, it is preferable to employ a clear plate). Thus, the dishes for each enzyme sample to be tested are (1) PBS only, (2) PBS and mannose-6-phosphate, (3) Enzyme, (4) Enzyme and mannose-6-phosphate, and (5) normal human fibroblasts. The dishes are incubated at 37° C. for 16 hours. Remove the medium and save in 15 ml conical tubes, wash the cells 3 times with DPBS, and harvest the cells with a cell scraper. Suspend cells in 1 ml DPBS and save in a 1.5 ml microcentrifuge tube. Centrifuge the cells at 14,000 RPM for 2 minutes, aspirate the DPBS and resuspend in 1 ml DPBS. Vortex the cells and repeat the centrifugation, and DPBS washing step 4 times. After the fourth washing step, lyse the cells in 110 μl 0.25% Triton X-100 at room temperature for 1 to 2 hours. The amount of protein present or the enzymatic activity is measured using the methods described herein or those commonly employed in the art.

The activity of GBA can be assayed using the following method. Prepare a 16 mM 4-methylumbelliferyl β-D-glucoside (4-MU-β-Glu) in 4×CP buffer (prepared by mixing 43.5 ml of 0.1M citric acid (21.01 g citric acid/liter) and 0.2 M Disodium Phosphate (28.4 g sodium phosphate anhydrous/liter)) mix and warm to 42° C. until the solution becomes clear. Then prepare an assay buffer by mixing equal parts of the 16 mM 4-MU-β-Glu and 4×CP Buffer and 1% TC/TX (made by dissolving, 1 gram of taurochoric acid, sodium salt in water and adjust volume to 90 ml, then add 10 ml of 10% Triton X-100). In a 96 well black pandex plate, add sample to assay (Adjust volume to 25 μl with water) and add 25 μl of assay buffer, incubate at 37° C. for 1 hour and stop the reaction by adding 125 mL of 1 M Glycine-NaOH, pH 10.5. Measure the amount of 4-MU released from 4-Mu-β-Glu by comparing fluorescence at Ex=360, Em=455 with a standard curve of free 4-MU.

To distinguish GBA activity from other types of beta glucocerbrosidase, other substrates, such as 4-MU-cellobioside, 4-MU-cellotrioside may be employed as well.

To determine the extent to which the GBA is phosphorylated, the GBA pre and post-phosphorylation treatment can be assayed by binding to Mannose-6-phosphate as described herein and in Hoflack et al (1985) J Bio Chem 260:12008-120014.

Recombinant expression vectors containing a nucleic acid sequence encoding GBA, GlcNAc phosphotransferase (soluble and insoluble forms) and/or phosphodiester α-GlcNAcase can be prepared using well-known techniques. The expression vectors include a DNA sequence operably linked to suitable transcriptional or translational regulatory nucleotide sequences such as those derived from mammalian, microbial, viral, or insect genes. Examples of regulatory sequences include transcriptional promoters, operators, enhancers, mRNA ribosomal binding sites, and appropriate sequences which control transcription and translation initiation and termination. Nucleotide sequences are “operably linked” when the regulatory sequence functionally relates to the DNA sequence for the appropriate enzyme. Thus, a promoter nucleotide sequence is operably linked to a GlcNAc-phosphotransferase DNA sequence if the promoter nucleotide sequence controls the transcription of the appropriate DNA sequence.

The ability to replicate in the desired host cells, usually conferred by an origin of replication and a selection gene by which transfectants are identified, may additionally be incorporated into the expression vector.

In addition, sequences encoding appropriate signal peptides that are not naturally associated with GBA, GlcNAc phosphotransferase (soluble and insoluble forms) and/or phosphodiester α-GlcNAcase can be incorporated into expression vectors. For example, a DNA sequence for a signal peptide (secretory leader) may be fused in-frame to the enzyme sequence so that the enzyme is initially translated as a fusion protein comprising the signal peptide. A signal peptide that is functional in the intended host cells enhances extracellular secretion of the appropriate polypeptide. The signal peptide may be cleaved from the polypeptide upon secretion of enzyme from the cell.

Suitable host cells for expression of the GBA, GlcNAc phosphotransferase and/or phosphodiester α-GlcNAcase include prokaryotes, yeast, archae, and other eukaryotic cells. Preferred cells include insect cells and eukaryotic cells, examples of which include, but not limited to: SF9, SF+, CHO, Hela, 293T NS0, etcetera.

Appropriate cloning and expression vectors for use with bacterial, fungal, yeast, and mammalian cellular hosts are well known in the art, e.g., Pouwels et al. Cloning Vectors: A Laboratory Manual, Elsevier, N.Y. (1985). The vector may be a plasmid vector, a single or double-stranded phage vector, or a single or double-stranded RNA or DNA viral vector. Such vectors may be introduced into cells as polynucleotides, preferably DNA, by well-known techniques for introducing DNA and RNA into cells. The vectors, in the case of phage and viral vectors also may be and preferably are introduced into cells as packaged or encapsulated virus by well-known techniques for infection and transduction. Viral vectors may be replication competent or replication defective. In the latter case viral propagation generally will occur only in complementing host cells. Cell-free translation systems could also be employed to produce the enzymes using RNAs derived from the present DNA constructs.

Expression vectors for use in host cells generally comprise one or more phenotypic selectable marker genes. A phenotypic selectable marker gene is, for example, a gene encoding a protein that confers antibiotic resistance or that supplies an autotrophic requirement. Examples of useful expression vectors for prokaryotic host cells include those derived from commercially available plasmids such as the cloning vector pBR322 (ATCC 37017). pBR322 contains genes for ampicillin and tetracycline resistance and thus provides simple means for identifying transfected cells. To construct an expression vector using pBR322, an appropriate promoter and a DNA sequence are inserted into the pBR322 vector.

Other commercially available vectors include, for example, pKK223-3 (Pharmacia Fine Chemicals, Uppsala, Sweden) and pGEM1 (Promega Biotec, Madison, Wis., USA).

Promoter sequences commonly used for recombinant prokaryotic host cell expression vectors include β-lactamase (penicillinase), lactose promoter system (Chang et al., Nature275:615, (1978); and Goeddel et al., Nature 281:544, (1979)), tryptophan (trp) promoter system (Goeddel et al., Nucl. Acids Res. 8:4057, (1980)), and tac promoter (Maniatis, Molecular Cloning: A Laboratory Manual, Cold Spring Harbor Laboratory, p. 412 (1982)).

Yeasts useful as host cells in the present invention include those from the genus Saccharomyces, Pichia, K. Actinomycetes and Kluyveromyces. Yeast vectors will often contain an origin of replication sequence from a 2μ yeast plasmid, an autonomously replicating sequence (ARS), a promoter region, sequences for polyadenylation, sequences for transcription termination, and a selectable marker gene. Suitable promoter sequences for yeast vectors include, among others, promoters for metallothionein, 3-phosphoglycerate kinase (Hitzeman et al., J. Biol. Chem. 255:2073, (1980)) or other glycolytic enzymes (Holland et al., Biochem. 17:4900, (1978)) such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase. Other suitable vectors and promoters for use in yeast expression are further described in Fleer et al., Gene, 107:285-195 (1991). Other suitable promoters and vectors for yeast and yeast transfectation protocols are well known in the art.

Yeast transfectation protocols are known to those of skill in the art. One such protocol is described by Hinnen et al., Proceedings of the National Academy of Sciences USA, 75:1929 (1978). The Hinnen protocol selects for Trp⁺ transfectants in a selective medium, wherein the selective medium consists of 0.67% yeast nitrogen base, 0.5% casamino acids, 2% glucose, 10 μg/ml adenine, and 20 μg/ml uracil.

Mammalian or insect host cell culture systems well known in the art could also be employed to express recombinant GBA, GlcNAc phosphotransferase and/or phosphodiester αa-GlcNAcase polypeptides, e.g., Baculovirus systems for production of heterologous proteins in insect cells (Luckow and Summers, Bio/Technology 6:47 (1988)) or Chinese hamster ovary (CHO) cells for mammalian expression may be used. Transcriptional and translational control sequences for mammalian host cell expression vectors may be excised from viral genomes. Commonly used promoter sequences and enhancer sequences are derived from Polyoma virus, Adenovirus 2, Simian Virus 40 (SV40), and human cytomegalovirus. DNA sequences derived from the SV40 viral genome may be used to provide other genetic elements for expression of a structural gene sequence in a mammalian host cell, e.g., SV40 origin, early and late promoter, enhancer, splice, and polyadenylation sites. Viral early and late promoters are particularly useful because both are easily obtained from a viral genome as a fragment which may also contain a viral origin of replication. Exemplary expression vectors for use in mammalian host cells are well known in the art.

The GBA, GlcNAc phosphotransferase and/or phosphodiester α-GlcNAcase of the present invention may, when beneficial, be expressed as a fusion protein that has the enzyme attached to a fusion segment. The fusion segment often aids in protein purification, e.g., by permitting the fusion protein to be isolated and purified by affinity chromatography. Fusion proteins can be produced by culturing a recombinant cell transfected with a fusion nucleic acid sequence that encodes a protein including the fusion segment attached to either the carboxyl and/or amino terminal end of the enzyme. Preferred fusion segments include, but are not limited to, glutathione-S-transferase, β-galactosidase, a poly-histidine segment capable of binding to a divalent metal ion, and maltose binding protein. In addition, the HPC-4 epitope purification system may be employed to facilitate purification of the enzymes of the present invention. The HPC-4 system is described in U.S. Pat. No. 5,202,253, the relevant disclosure of which is herein incorporated by reference.

According to the present invention, isolated enzymes may be produced by the recombinant expression systems described above. The method comprises culturing a host cell transfected with an expression vector comprising a DNA sequence that encodes the enzyme under conditions sufficient to promote expression of the enzyme. The enzyme is then recovered from culture medium or cell extracts, depending upon the expression system employed. As is known to the skilled artisan, procedures for purifying a recombinant protein will vary according to such factors as the type of host cells employed and whether or not the recombinant protein is secreted into the culture medium. When expression systems that secrete the recombinant protein are employed, the culture medium first may be concentrated. Following the concentration step, the concentrate can be applied to a purification matrix such as a gel filtration medium. Alternatively, an anion exchange resin can be employed, e.g., a matrix or substrate having pendant diethylaminoethyl (DEAE) groups. The matrices can be acrylamide, agarose, dextran, cellulose, or other types commonly employed in protein purification. Also, a cation exchange step can be employed. Suitable cation exchangers include various insoluble matrices comprising sulfopropyl or carboxymethyl groups. Further, one or more reversed-phase high performance liquid chromatography (RP-HPLC) steps employing hydrophobic RP-HPLC media (e.g., silica gel having pendant methyl or other aliphatic groups) can be employed to further purify the enzyme. Some or all of the foregoing purification steps, in various combinations, are well known in the art and can be employed to provide an isolated and purified recombinant protein.

Recombinant protein produced in bacterial culture is usually isolated by initial disruption of the host cells, centrifugation, extraction from cell pellets if an insoluble polypeptide, or from the supernatant fluid if a soluble polypeptide, followed by one or more concentration, salting-out, ion exchange, affinity purification, or size exclusion chromatography steps. Finally, RP-HPLC can be employed for final purification steps. Host cells can be disrupted by any convenient method, including freeze-thaw cycling, sonication, mechanical disruption, or use of cell lysing agents.

The invention provides methods of phosphorylating GBA and the thus obtained phosphorylated GBA enzymes. GBA is produced by treating the high mannose GBA with α/β GlcNAc-phosphotransferase (soluble or insoluble, as well as mixed α/β/γ GlcNAc phosphotransferase, with α/β GlcNAc phosphotransferase) which catalyzes the transfer of N-acetylglucosamine-1-phosphate from UDP-GlcNAc to the 6′ position of 1,2-linked or other mannoses on the hydrolase. Preferably, the GlcNAc-phosphotransferase is the soluble α/β GlcNAc-phosphotransferase described herein. Also it was shown that the γ subunit of the GlcNAc-phosphotransferase is not required.

Methods for treating GBA with the enzymes of the present invention are within the skill of the artisan. Generally, the GBA is at a concentration of about 10 mg/ml and GlcNAc-phosphotransferase is present in a concentration of about 1 to about 10 million units per milliliter. The enzymes are incubated at about 20° C. for about 48 hours or longer in the presence of a buffer that maintains the pH at about 6-7 and any stabilizers or coenzymes required to facilitate the reaction. Then, phosphodiester α-GlcNAcase can be added to the system to a concentration of about 250,000 to 1,000,000 units/mL and the system is allowed to incubate for about 6 or more hours. The modified GBA enzyme having highly phosphorylated oligosaccharides is then recovered by conventional means.

In a preferred embodiment, the GBA at 10 mg/ml is incubated in 50 mm Sodium Acetate pH 6.5, 20 mM MnCl₂, 0.3 mM (300 μM) with GlcNAc phosphotransferase at 1 to 10 million units/ml at 20° C. for 48 hours or longer,. The GBA is then treated with phosphodiester-α GlcNAcase for 6 hours. The modified enzyme is then repurified by conventional chromatography.

High mannose GBA for treatment according to the present invention can be obtained from any convenient source, e.g., by isolating and purifying naturally occurring enzymes or by recombinant techniques for the production of proteins.

High mannose GBA can be prepared by expressing the DNA encoding the GBA in any host cell system that generates a oligosaccharide modified protein having high mannose structures, e.g., yeast cells, insect cells, other eukaryotic cells, transfected Chinese Hamster Ovary (CHO) host cells, or other mammalian cells.

In one embodiment, high mannose GBA is produced using mutant yeast that are capable of expressing peptides having high mannose structures. These yeast include the mutant S. cervesiae ochl, mnnl (Nakanishi-Shindo, Y., Nakayama, K. I., Tanaka, A., Toda, Y. and Jigami, Y. (1993). “Structure of the N-linked oligosaccharides that show the complete loss of α-1,6-polymannose outer chain from ochl, ochl mnnl, and ochl mnnl alg3 mutants of Saccharomyces cerevisiae.” Journal of Biological Chemistry 268: 26338-26345).

Preferably, high mannose GBA is produced using over-expressing transfected insect, CHO, or other mammalian cells that are cultured in the presence of certain inhibitors. Normally, cells expressing lysosomal enzymes secrete enzymes that contains predominantly sialylated complex type glycans that do not serve as a substrate for GlcNAc-phosphotransferase and therefore cannot be modified to use the M6P receptor.

According to the present invention, transfected cells containing DNA that expresses a recombinant GBA can be manipulated so that the cells secrete high mannose GBA that can be modified according to the above method. In this method, transfected cells are cultured in the presence of α 1,2-mannosidase inhibitors and the high mannose recombinant GBA is recovered from the culture medium. Inhibiting α 1,2-mannosidase prevents the enzyme from trimming mannoses and forces the cells to secrete glycoproteins having the high mannose structure. High mannose GBA is recovered from the culture medium using known techniques and treated with α/β GlcNAc-phosphotransferase and phosphodiester α-GlcNAcase according to the method herein to produce GBA that has M6P and can therefore bind to membrane M6P receptors and be taken into the cell having the M6P receptor. Preferably, the cells are CHO cells and the GBA is secreted with the MAN7(D₂D₃) structure, more preferably the cells are CHO K1 cells, and even more preferred are CHO K1 cells that are GnT I deficient.

In a preferred embodiment, recombinant human GBA is prepared by culturing CHO cells secreting GBA in Dulbecco's modified essential media (DMEM) modified by the addition of an alpha 1,2-mannosidase inhibitor. Isolation of GBA from the media followed by digestion with either N-glycanase or endoglycosidase-H demonstrates that in the presence of the alpha 1,2-mannosidase inhibitor the GBA retains high mannose structures rather than the complex structures found on a preparation secreted in the absence of the inhibitor. The isolated GBA bearing high mannose structures is then purified to homogeneity, preferably by chromatography beginning with ion exchange chromatography on ConA-Sepharose, followed toyopearl butyl 650M, Phenyl-Sepharose or octyl Sepharose. The purified GBA is then treated in vitro with α/βGlcNAc-phosphotransferase to convert specific mannoses to GlcNAc-phospho-mannose diesters. The GlcNAcphosphomannose diesters are then converted to M6P groups by treatment with phosphodiester a GlcNAcase.

Any α1,2-mannosidase inhibitor can function in the present invention. Preferably, the inhibitor is selected from the group consisting of deoxymannojirimycin (dMM), kifunensine, D-Mannonolactam amidrazone, and N-butyl-deoxymannojirimycin. Most preferably the inhibitor is deoxymannojimycin and/or kifunensine.

The present invention also provides methods for the treatment of Gaucher's disease by administering an effective amount of the highly phosphorylated GBA of the present invention to a patient diagnosed with the Gaucher's disease. As used herein, being diagnosed with Gaucher's includes pre-symptomatic phases of the disease and the various symptomatic Gaucher's disease. Typically, the pre-symptomatic patient will be diagnosed with Gaucher's disease by means of a genetic analysis known to the skilled artisan.

As discussed above, the administration of the highly phosphorylated GBA will target tissues, for example, lung and bone tissues, that posses the M6P receptor. Thus, the present highly phosphorylated GBA will be targeted to tissues that are not normally targeted using recombinant or naturally purified GBA thereby resulting in an increased positive effect on the patient suffering from Gaucher's disease.

In one embodiment of the present invention is a method of treating lung or lung tissue in patients with Gaucher's by administering the highly phosphorylated GBA to said patient. In another embodiment of the present invention the highly phosphorylated GBA is administered to bone or bone tissue of Gaucher's patients.

In one embodiment, the present invention provides a method of treating Gaucher's disease using a combination of both highly phosphorylated GBA prepared in accordance with the present invention and the GBA not so prepared, i.e., having little or no phosphorylation. Therefore, the combination of both types of GBA enzymes would substantially increase the tissues treated by the enzyme replacement therapy as described herein.

While dosages may vary depending on the disease and the patient, highly phosphorylated GBA is generally administered to the patient in amounts of from about 0.1 to about 1000 milligrams per kg of patient per month, preferably from about 1 to about 500 milligrams per kg of patient per month. The highly phosphorylated GBA of the present invention is taken into the cell expressing the M6P receptor than the naturally occurring or less phosphorylated GBA and are therefore effective for the treatment of Gaucher's disease. Amongst various patients the severity and the age at which the disease presents itself may be a function of the amount of residual GBA enzyme that exists in the patient. As such, the present method of treating Gaucher's diseases includes providing the highly phosphorylated GBA at any or all stages of disease progression.

The GBA enzyme may be administered by any convenient means, conventionally known to those of ordinary skill in the art. For example, the enzyme may be administered in the form of a pharmaceutical composition containing the enzyme and a pharmaceutically acceptable carrier or by means of a delivery system such as a liposome or a controlled release pharmaceutical composition. The term “pharmaceutically acceptable” refers to molecules and compositions that are physiologically tolerable and do not typically produce an allergic or similar unwanted reaction such as gastric upset or dizziness when administered. Preferably, “pharmaceutically acceptable” means approved by a regulatory agency of the Federal or a state government or listed in the U.S. Pharmacopoeia or other generally recognized pharmacopoeia for use in animals, preferably humans. The term “carrier” refers to a diluent, adjuvant, excipient, or vehicle with which the compound is administered. Such pharmaceutical carriers can be sterile liquids, such as saline solutions, dextrose solutions, glycerol solutions, water and oils emulsions such as those made with oils of petroleum, animal, vegetable, or synthetic origin (peanut oil, soybean oil, mineral oil, or sesame oil). Water, saline solutions, dextrose solutions, and glycerol solutions are preferably employed as carriers, particularly for injectable solutions.

The enzyme or the composition may be administered by any standard technique compatible with enzymes or their compositions. For example, the enzyme or composition can be administered parenterally, transdermally, or transmucosally, e.g., orally or nasally. Preferably, the enzyme or composition is administered by intravenous injection.

The following Examples provide an illustration of embodiments of the invention and should not be construed to limit the scope of the invention which is set forth in the appended claims. In the following Examples, all methods described are conventional unless otherwise specified.

### EXAMPLES

Human Acid β-Glucocerbrosidase

A mammalian expression vector was constructed by subcloning a cDNA for human acid β-glucocerbrosidase (GBA) into the EcoR I site of the pcDNA6/V5/His-A (Invitrogen) construct. This plasmid was renamed pDH1. A cDNA of human GBA was subcloned into the XbaI and EcoRI sites of the pEE14 vector (Lonza Biologics) and named pCC4.

Human GlcNAc-Phosphotransferase

Plasmid pMK 163 was constructed to express recombinant soluble human GlcNAc-phosphotransferase. GlcNAc-phosphotransferase is an enzyme that consists of four subunits; α2β2. The α and β subunits are encoded on a single mRNA and proteolytically cleaved after translation. The wild type human GlcNAc-phosphotransferase is associated with the Golgi apparatus through transmembrane domains at the N-terminal of the α subunit, and C-terminal of the β subunit. By the following modification, a cDNA encoding a soluble form of recombinant human GlcNAc-phosphotransferase was made. The α/β subunit was modified from the wild type sequence as follows; (1) 24 amino acids located on the N-terminal of the α subunit, which is a putative signal/transfer transmembrane domain, were replaced with the immunoglobulin leader sequence (METDTLLLWVLLLWVPGSTG-SEQ ID NO:22) and the HPC4 epitope (DEDQVDPRLIDGK-SEQ ID NO:23)(Rezaie, A. R et. al (1992) Protein Expr Purif, 3, 453-60) (2) 47 amino acids at the C-terminus were removed by replacing the codons encoding these amino acids by a stop codon. (3) 6 amino acids just before the α/β cleavage site were replaced with RARYKR (SEQ ID NO:27)which is a cleavage sequence for furin (Nakayama, K., (1997) Biochem.J, 327, 625-635), which is a proprotein processing enzyme. The plasmid pMK 155 uses a pEE14 (Lonza Biologics) backbone to express α/β subunits, thus modified.

Human N-acetylglucosamine-1-Phosphodiester α-N-acetylglucosaminidase (UCE)

Plasmid pKB 6 was constructed to express recombinant soluble uncovering enzyme. The molecular cloning and expression of wild type uncovering enzyme is described in Kornfeld et al. ((1999) Biochem J, 274, 32778-32785). Uncovering enzyme consists of four identical subunits arranged as two disulfide-linked homodimers. The wild type human uncovering enzyme is associated with the Golgi apparatus through a transmembrane domain at the C-terminal end of the polypeptide. A cDNA encoding a soluble form of recombinant human uncovering enzyme was made by replacing 68 amino acids at the C-terminal with a HPC4 epitope tag (EDQVDPRLIDGKD-(SEQ ID NO:3)). The modified cDNA encoding soluble rh-UCE then was subcloned into pEE 14 (Lonza Biologics).

GBA Transfection

The cells were cultured in 16% CO2 to maintain a slightly acidic culture medium. In order to express the GBA protein the pDH1 plasmid was transienty transfected into 293T cells. Four Nunc cell factories (6320 cm²) were seeded with approximately 2×10⁸ cells each in Dulbecco's Modification of Eagles Medium (DMEM) containing 10% fetal bovine serum (FBS). In addition kifunensine, a glycosidase inhibitor which acts on the N-linked oligosaccharides processing pathway was added to 5 μg/ml. The cells were transfected using FuGene 6 (Roche) according to the manufacturers instruction. The media from the cells were harvested approximately 96 hours post-transfection.

GlcNAc-phosphotransferase Transfection

In order to develop a stable cell line secreting soluble human GlcNAc-phosphotransferase, the glutamine synthetase (GS) expression system (Lonza Biologics) was utilized (Bebbington, C. R., (1998) Current Protocols in Molecular Biology, 16(14),7-13). The pMK 155 plasmid was transfected into CHO-K1 cells and the media from clones which survived the methionine sulfoximine (MSX) selection were assayed for GlcNAc-phosphotransferase activity (Reitman, M. L., et.al (1984) Methods Enzymol. 107, 163-172). A clone expressing high levels of GlcNAc-phosphotransferase in the media was selected as the source of GlcNAc-phosphotransferase. In addition, a stable line using pCC4 was made under the same conditions as described above.

Phosphodiester α-GlcNAcase Transfection

In order to develop a stable cell line secreting soluble human uncovering enzyme, glutamine synthetase (GS) system (Lonza Biologics) was utilized. The plasmid pKB 6 was transfected into CHO-K1 cells and the media from clones which survived the MSX selection were assayed for UCE activity (9). A clone expressing high levels of UCE was selected as the source of uncovering enzyme.

Purification of GBA

The GBA purification scheme consisted of concentrating the harvested media 10-fold from 8 liters to 0.8 liters with a Millipore Pelicon concentrator and then incubating the concentrated media with Con A sepharose (Pharmacia) for approximately 3 hours at 4° C. The Con A sepharose was then packed into a column, washed with 25 mM Tris-HCl pH 6.5, 0.5M NaCl, 1 mM MnCl₂, 1 mM CaCl₂ and eluted with 25 mM Tris-HCl, pH 6.5, 0.5 M NaCl, 1M α-methyl glucoside. The fractions were assayed for GBA and the peak fractions were pooled. The GBA was then loaded onto a Toyopearl Butyl 650M (TosoHass) column and eluted with a 10 column volume gradient of 0-60% ethylene glycol followed by 100% ethylene glycol. The fractions were again assayed for GBA activity. The peak fractions were pooled and dialyzed overnight at 4° C. in 50 mM sodium acetate pH 5.5, 150 mM NaCl.

GBA activity was measured by using 4-methyl-umberyferyl-β-glucoside (4MU-β-Glc, Sigma) as a substrate. The amount of GBA which converts 1 nmol of 4MU-β-Glc into 1 .nmol each of 4-methyl-umberyferone (4MU) and glucoside at 37° C. per hour was defined as 1 unit.

GBA

4MU-β-Glc→4MU+Glc

Briefly, samples were incubated with 4 mM 4MU-β-Glc in 1×assay buffer in the presence of 0.25% (V/V) Triton X-100 and 0.25% (W/V) sodium taurocholate in 40 μl at 37° C. for 30 min to 2 hrs. A 4×assay buffer, pH 5.5, was made by mixing 43.5 ml of 0.1 M citric acid and 0.2 M disodium phosphate solution. The reaction was stopped by adding 100 μl of 1 M Glycine/NaOH, pH 10.5 solution. The amount of 4-MU converted. during the incubation was measured by detecting fluorescence at excitation wavelength=360 nm, emission wavelength=455 nm. Assay results were compared to a standard curve obtained from known amount (0, 25, 50, 100, 200, and 400 pmol) of 4-MU (Sigma).

Purification of GlcNAc-phosphotransferase

HPC4 (Oklahoma Medical Research Foundation; OMRF) was coupled to Ultralink Biosupport Medium (Pierce) following the manufacturer's instructions. The HPC4:ultralink resin was equilibrated in 50 mM Tris-HCl, 150 mM NaCl and 2 mM CaCl₂. Cell culture medium from the CHO-K1 cells was incubated with the HPC4:ultralink resin for 16 hrs at 4° C. to capture the GlcNAc-phosphotransferase which contained the epitope tag for the HPC4. The bound GlcNAc-phosphotransferase was eluted with 50 mM Tris-HCl, 150 mM NaCl and 5 mM EGTA, concentrated and buffer-exchanged to 50 mM Tris-HCl, 150 mM NaCl and 5 mM MgCl₂. The amount of GlcNAc-phosphotransferase which can transfer 1 pmol per hour of GlcNAc-phosphate from UDP-GlcNAc (donor) to α-methyl mannoside (acceptor) is defined as 1 unit (Reitman et al Meth. Enzym. 107:163-172 (1984)).

Purification of phosphodiester α-GlcNAcase

HPC4 (OMRF) was coupled to Ultralink Biosupport Medium (Pierce) following the manufacturer's instructions. The HPC4:ultralink resin was equilibrated in 50 mM Tris-HCl, 150 mM NaCl and 2 mM CaCl₂. Cell culture medium from the transfected CHO-K1 cells was incubated with the HPC4:ultralink resin for 16 hrs at 4° C. to capture the phosphodiester α-GlcNAcase which contained the epitope tag for HPC4. The the phosphodiester α-GlcNAcase was eluted with 50 mM Tris-HCl, 150 mM NaCl and 5 mM EDTA and. Recombinant human the phosphodiester α-GlcNAcase thus prepared was used for uncovering of phosphorylated acid-β-glucocerbrosidase. The amount of the phosphodiester α-GlcNAcase that can remove 1 nmol of GlcNAc per hour from GlcNAc-α-P-ManαMe is defined as 1 unit (Mullis, K., et al (1994) Biochem. J, 269, 1718-1726).

Preparation of Highly Phosphorylated GBA

Partially purified GBA (1462 units) was phosphorylated by incubating with GlcNAc-phosphotransferase (100,000 unit) in 50 mM sodium acetate (pH 6.5), 20 mM MgCl₂, and 150 μM UDP-GlcNAc at 20° C. for 47 hrs. Next, 1000 units of uncovering enzyme and a phosphatase inhibitor cocktail II (Sigma) were added and the reaction was incubated an additional 6.5 hours at 20° C. Following the uncovering reaction β-glycerophosphate was added to 5 mM to inhibit phosphatase activity. Next, the HP-GBA was examined for its binding efficiency to a mannose-6-phosphate receptor column.

Mannose-6-Phosphate Receptor (M6P-R) Binding Pre- and Post-Phosphorylation

Mannose 6-phosphate (M6P) receptor was purified from bovine liver and coupled to a NHS-Sepharose 4B FF resin (Hoflack et al (1985) J Bio Chem 260:12008-120014). The resin was then packed in a 2 ml column and equilibrated with a buffer consisting of 50 mM Imidazole, 150 mM NaCl, 2 mM EDTA, 5 mM β-glycerophosphate, 0.05% v/v Triton X-100, 0.02% v/v sodium azide, at a flow rate of 0.1 ml/min. The GBA was injected onto the M6PR column and then a linear gradient of increasing M6P was applied after the column had been washed with 5.5 column volumes of the buffer mentioned above. A gradient, 0-1 mM M6P was allowed to develop over the next 10 ml at which time M6P was increased to 5 mM and maintained for 5 mls. At this time the column was returned to its initial conditions. During the entire chromatograph, 250 μl fractions were collected and subsequently assayed for GBA activity. The fluorescence of each well was then graphed and overlaid with the M6P gradient applied to the column. The elution of GBA is positively correlated to the amount of phosphorylated mannose present on the enzyme.

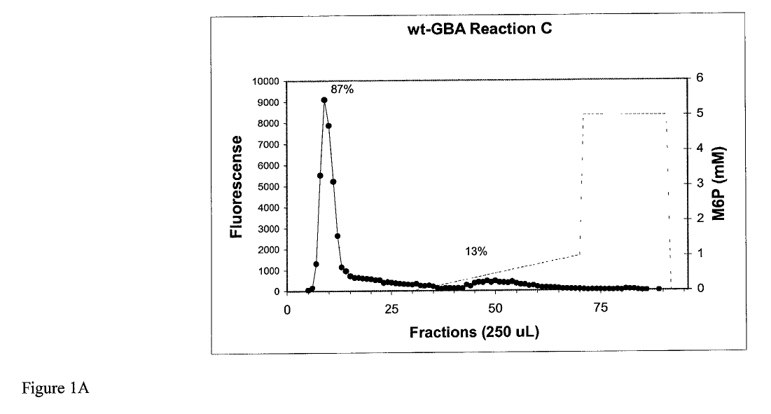

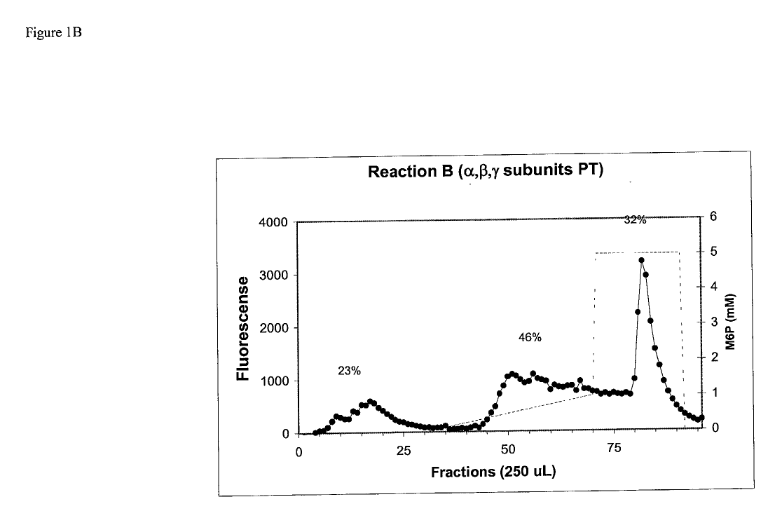

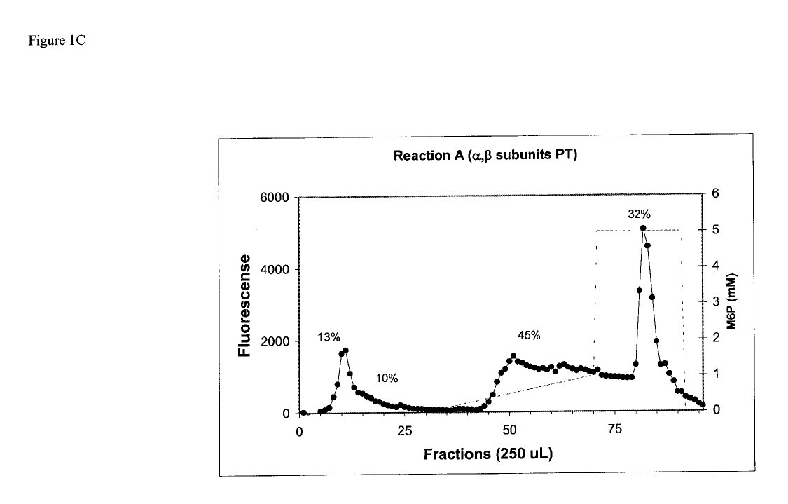

Following the GBA purification, a aliquot (˜250 U) was loaded onto a M6PR column, a M6P gradient was run, fractions collected and GBA activity assayed. As illustrated in FIG. 1A over 99% of the sample was eluted prior to the start of the M6P gradient suggesting there was no detectable phosphorylated GBA. As illustrated in FIG. 1B, there was a pronounced shift in the elution of phosphorylated GBA. Seventy-seven percent of the GBA activity was eluted after the start of the M6P gradient. Of that, 35% was eluted only after the addition of 5 mM M6P which suggest a phosphorylated GBA molecule.

GBA was poorly phosphorylated under culture conditions. However, upon treatment with GlcNAc phosphotransferase and phosphodiester α-GlcNAcase, a highly phosphorylated GBA was obtained.

Obviously, numerous modifications and variations on the present invention are possible in light of the above teachings. It is therefore to be understood that within the scope of the appended claims, the invention may be practiced otherwise than as specifically described herein.

What is claimed:

1. A method of preparing a highly phosphorylated acid β-glucocerebrosidase comprising:
(a) contacting an acid β-glucocerebrosidase with an isolated GlcNAc phosphotransferase to produce a modified acid β-glucocerebrosidase; and
(b) contacting said modified acid β-glucocerebrosidase with an isolated phosphodiester α-GlcNAcase.

2. The method of claim 1, further comprising purifying said highly phosphorylated acid β-glucocerebrosidase after said contacting with the isolated phosphodiester α-GlcNAcase.

3. The method of claim 1, further comprising purifying said modified acid β-glucocerebrosidase prior to said contacting with the isolated phosphodiester α-GlcNAcase.

4. The method of claim 1, wherein said isolated GlcNAc phosphotransferase comprises an α subunit and β subunit.

5. The method of claim 4, wherein the GlcNAc phosphotranferase comprises the amino acid of SEQ ID NO:2.

6. The method of claim 1, wherein the GlcNAc phosphotranferase comprises SEQ ID NO:4 and SEQ ID NO:5.

7. The method of claim 1, wherein the GlcNAc phosphotranferase is encoded by a nucleotide sequence comprising SEQ ID NO:1, or a sequence that hybridizes under stringent conditions to SEQ ID NO:1.

8. The method of claim 1, The method of claim 1, wherein the GlcNAc phosphotranferase is encoded by a nucleotide sequence comprising SEQ ID NO:3, or a sequence that hybridizes under stringent conditions to SEQ ID NO:3.

9. The method of claim 1, wherein the phosphodiester α-GlcNAcase comprises SEQ ID NO:17 or a sequence that hybridizes under stringent conditions to SEQ ID NO:17.

10. The method of claim 1, wherein the acid β-glucocerebrosidase comprises the amino acid sequence of SEQ ID NO:25 or SEQ ID NO:26.

11. The method of claim 1, wherein the acid-β-glucocerebrosidase comprises the amino acid sequence of SEQ ID NO:26.

12. A highly phosphorylated acid β-glucocerebrosidase obtained by the method of claim 1.

13. A pharmaceutical composition comprising the highly phosphorylated acid β-glucocerebrosidase of claim 12 and a pharmaceutically acceptable carrier.

14. A method of treating a patient suffering from Gaucher's disease, comprising administering to the patient in need thereof the highly phosphorylated acid beta-glucocerebrosidase of claim 12 in an amount sufficient to treat said disease.

15. The method of claim 14, further comprising administering acid β-glucocerebrosidase which is not highly phosphorylated.

16. A method of treating a bone tissue of a patient suffering from Gaucher's disease, comprising administering to the patient in need thereof the highly phosphorylated acid beta-glucocerebrosidase of claim 12 in an amount sufficient to treat said disease.

17. The method of claim 16, further comprising administering acid β-glucocerebrosidase which is not highly phosphorylated.

18. A method of treating a lung tissue of a patient suffering from Gaucher's disease, comprising administering to the patient in need thereof the highly phosphorylated acid beta-glucocerebrosidase of claim 12 in an amount sufficient to treat said disease.

19. The method of claim 18, further comprising administering acid β-glucocerebrosidase which is not highly phosphorylated.

20. A method of producing a highly phosphorylated acid β-glucocerebrosidase comprising:
(a) culturing transfected cells comprising a recombinant polynucleotide which encodes a recombinant acid β-glucocerebrosidase in the presence of at least one α 1,2-mannosidase inhibitor;
(b) recovering a high mannose recombinant acid β-glucocerebrosidase from said transfected cell;
(c) contacting said high mannose recombinant acid β-glucocerebrosidase with an isolated GlcNAc phosphotransferase to produce a modified acid β-glucocerebrosidase; and
(d) contacting said modified acid β-glucocerebrosidase with an isolated phosphodiester α-GlcNAcase.

21. The method of claim 20, wherein said at least one 1,2-mannosidase inhibitor is selected from the group consisting of deoxymannojirimycin, kifunensine, D-Mannonolactam amidrazone, and N-butyl-deoxymannojirimycin.

22. The method of claim 21, wherein the 1,2-mannosidase inhibitor is kifunensine.

23. The method of claim 21, wherein the 1,2 mannosidase inhibitor is deoxymannojirimycin.

24. The method of claim 21, wherein the at least one 1,2 mannosidase inhibitor is deoxymannojirimycin and kifunensine.

25. The method of claim 20, further comprising purifying said modified acid β-glucocerebrosidase prior to said contacting with the isolated phosphodiester α-GlcNAcase.

26. The method of claim 20, wherein said isolated GlcNAc phosphotransferase comprises an α subunit and β subunit.

27. The method of claim 26, wherein the GlcNAc phosphotranferase comprises the amino acid SEQ ID NO:2.

28. The method of claim 20, wherein the GlcNAc phosphotranferase comprises SEQ ID NO:4 and SEQ ID NO:5.

29. The method of claim 20, wherein the GlcNAc phosphotranferase is encoded by a nucleotide sequence comprising SEQ ID NO:1, or a sequence that hybridizes under stringent conditions to SEQ ID NO:1.

30. The method of claim 20, The method of claim 1, wherein the GlcNAc phosphotranferase is encoded by a nucleotide sequence comprising SEQ ID NO:3, or a sequence that hybridizes under stringent conditions to SEQ ID NO:3.

31. The method of claim 20, wherein the phosphodiester α-GlcNAcase comprises SEQ ID NO:17 or a sequence that hybridizes under stringent conditions to SEQ ID NO:17.

32. The method of claim 20, wherein the acid β-glucocerebrosidase comprises the amino acid sequence of SEQ ID NO:25 or SEQ ID NO:26.

33. The method of claim 20, wherein the acid-β-glucocerebrosidase comprises the amino acid sequence of SEQ ID NO:26.

34. A highly phosphorylated acid β-glucocerebrosidase obtained by the method of claim 20.

35. A pharmaceutical composition comprising the highly phosphorylated acid β-glucocerebrosidase of claim 34 and a pharmaceutically acceptable carrier.

36. A method of treating a patient suffering from Gaucher's disease, comprising administering to the patient in need thereof the highly phosphorylated acid beta-glucocerebrosidase of claim 34 in an amount sufficient to treat said disease.

37. The method of claim 36, further comprising administering acid β-glucocerebrosidase which is not highly phosphorylated.

38. A method of treating a bone tissue of a patient suffering from Gaucher's disease, comprising administering to the patient in need thereof the highly phosphorylated acid beta-glucocerebrosidase of claim 34 in an amount sufficient to treat said disease.

39. The method of claim 38, further comprising administering acid β-glucocerebrosidase which is not highly phosphorylated.

40. A method of treating a lung tissue of a patient suffering from Gaucher's disease, comprising administering to the patient in need thereof the highly phosphorylated acid beta-glucocerebrosidase of claim 34 in an amount sufficient to treat said disease.

41. The method of claim 40, further comprising administering acid β-glucocerebrosidase which is not highly phosphorylated.

42. A highly phosphorylated acid β-glucocerebrosidase, which is encoded by the nucleotide sequence of SEQ ID NO:24 of a nucleotide sequence that hybridizes to the nucleotide sequence of SEQ ID NO:24.

43. The highly phosphorylated acid β-glucocerebrosidase of claim 28, which comprises the amino acid sequence of SEQ ID NO:25 or SEQ ID NO:26.

44. The highly phosphorylated acid β-glucocerebrosidase of claim 43, which comprises the amino acid sequence of SEQ ID NO:26.

45. A pharmaceutical composition comprising the highly phosphorylated acid β-glucocerebrosidase of claim 42 and a pharmaceutically acceptable carrier.

46. A method of treating a patient suffering from Gaucher's disease, comprising administering to the patient in need thereof the highly phosphorylated acid beta-glucocerebrosidase of claim 42 in an amount sufficient to treat said disease.

47. The method of claim 46, further comprising administering acid β-glucocerebrosidase which is not highly phosphorylated.

48. A method of treating a bone tissue of a patient suffering from Gaucher's disease, comprising administering to the patient in need thereof the highly phosphorylated acid beta-glucocerebrosidase of claim 42 in an amount sufficient to treat said disease.

49. The method of claim 48, further comprising administering acid β-glucocerebrosidase which is not highly phosphorylated.

50. A method of treating a lung tissue of a patient suffering from Gaucher's disease, comprising administering to the patient in need thereof the highly phosphorylated acid beta-glucocerebrosidase of claim 42 in an amount sufficient to treat said disease.

51. The method of claim 50, further comprising administering acid β-glucocerebrosidase which is not highly phosphorylated.

52. A method of preparing a highly phosphorylated acid β-glucocerebrosidase comprising:
(i) a step for transferring a N-acetylglucosamine-1-phosphate from UDP-GlcNAc to an acid β-glucocerebrosidase;
(ii) a step for removing an N-acetylglucosamine from said acid β-glucocerebrosidase.

53. A highly phosphorylated acid β-glucocerebrosidase obtained by the method of claim 52.

54. The highly phosphorylated acid β-glucocerebrosidase of claim 53, which comprises the amino acid sequence of SEQ ID NO:26.

55. A pharmaceutical composition comprising the highly phosphorylated acid β-glucocerebrosidase of claim 53 and a pharmaceutically acceptable carrier.

56. A method of treating a patient suffering from Gaucher's disease, comprising administering to the patient in need thereof the highly phosphorylated acid beta-glucocerebrosidase of claim 53 in an amount sufficient to treat said disease.

57. The method of claim 56, further comprising administering acid β-glucocerebrosidase which is not highly phosphorylated.

58. A method of treating a bone tissue of a patient suffering from Gaucher's disease, comprising administering to the patient in need thereof the highly phosphorylated acid beta-glucocerebrosidase of claim 53 in an amount sufficient to treat said disease.

59. The method of claim 58, further comprising administering acid β-glucocerebrosidase which is not highly phosphorylated.

60. A method of treating a lung tissue of a patient suffering from Gaucher's disease, comprising administering to the patient in need thereof the highly phosphorylated acid beta-glucocerebrosidase of claim 53 in an amount sufficient to treat said disease.

61. The method of claim 60, further comprising administering acid β-glucocerebrosidase which is not highly phosphorylated.
